(19) United States
(12) Patent Application Publication
(10) Pub. No.: US 2005/0203457 A1
(43) Pub. Date: Sep. 15, 2005

(54) Apparatus and method for creating an arterio-venous connection in hemodialysis maintenance

(76) Inventor: Douglas R. Smego, Greenwich, CT (US)

Correspondence Address: David Prashker;DAVID PRASHKER, P.C., P.O. Box 5387, Magnolia, MA, 01930, US

(21) Appl. No.: 11/074,384

(22) Filed: Mar. 7, 2005

Related U.S. Application Data
(60) Provisional application No. 60/553,007, filed on Mar. 15, 2004.

(51) Int. Cl. A61M 5/00

(52) U.S. Cl. 604/8; 623/1.1; 623/1.23; 606/153

(57) ABSTRACT

The present invention provides a kit apparatus and a methodology to prevent the primary causes of arterio-venous graft thrombosis; and provides a durable vascular access for successful long term use in hemodialysis. The invention employs a patient-customized prosthetic endograft as an subcutaneously implanted vascular access; and utilizes a surgical method for endovascular insertion of the prosthetic endograft into a pre-chosen vein, which does not require a distal anastomosis, and thus allows the distal outflow end of the implanted vascular access to remain unattached and freely floating at a precisely located anatomic position within the internal lumen the pre-chosen vein.

### PRIORITY CLAIM

This invention was first filed as U.S. Provisional Patent Application No. 60/553,007 on Mar. 15, 2004. The filing date and priority of this first filing is expressly claimed pursuant to 35 U.S.C. 119(e).

### FIELD OF THE INVENTION

This invention relates generally to the making of a permanent anatomic connection to access the vascular blood system in-vivo; and is directed specifically to apparatus and methods for creating a vascular access suitable for blood dialysis in humans afflicted with end stage renal disease (or “ESRD”).

### BACKGROUND OF THE INVENTION

Renal disease continues to be an important cause of mortality and morbidity in the United States and throughout the world. Renal disease may be acute or chronic. Acute renal failure is a worsening of renal function over hours to days, resulting in the retention of nitrogenous wastes (such as urea nitrogen) and creatinine in the blood. In comparison, chronic renal failure results from a loss of renal function over months to years. It is presently estimated that between 4-5% of the entire American population have some form of kidney disease; and that over four hundred thousand persons in America reach that life threatening medical condition or clinical stage known as End Stage Renal Disease (hereinafter “ESRD”) which signifies the complete lack of life preserving renal function for the kidneys in that person.

Based upon 2002 data from the CMS, the National Kidney Foundation and the End Stage Renal Disease Network, there are approximately 406,000 patients with end stage renal disease in the United States. In 1990, the same sources utilizing the same definitions and processes estimated just over 200,000 patients with end stage renal disease. Unquestionably, there has been a constant increase in the number of patients with renal disease of some variety, now estimated at 4.45% of the entire population.

The largest percentages increases have been seen in the group of patients requiring treatment for end stage renal disease; and it is the elderly population which has seen the largest increases in renal disease and in end stage renal disease particularly. The rate of increase, as seen from the overall number of new cases reported each year, has gone from just over fifty thousand per year to ninety thousand per year over the decade from 1991 through 2001. Of those persons currently afflicted with end stage renal disease, it is estimated that 293,000 (72%) are currently receiving hemodialysis treatment, thus averaging 348 hemodialysis patients per million in the U.S. population.

### A. End Stage Renal Disease

Persons suffering from End Stage Renal Disease constitute a particular class of medical patients which require renal replacement therapy, either in the form of blood dialysis or kidney transplantation, in order to survive. A healthy kidney functions to remove toxic wastes and excess water from the blood. However, with End Stage Renal Disease (“ESRD”), there is chronic kidney failure; and the kidneys progressively fail and stop performing their essential functions over an extended period of time. If and when the kidneys progressively continue to fail in this manner, the patient afflicted with ESRD will die within a short period of time (usually hours or days) unless (I) that patient receives blood dialysis treatment quickly, a process which must then be continued and repeatedly performed at regular time intervals for the rest of that patient's life; or (ii) the patient undergoes transplantation therapy and receives a healthy and biocompatible, normal kidney from a donor. Unfortunately, because relatively few kidneys are presently available for transplantation purposes, the overwhelming majority of patients suffering from ESRD must receive regular blood dialysis treatments for the remainder of their lives.

It will be recognized also that the present rate of human ESRD is more than twice the incidence rate reported ten years ago, with more than ninety thousand new ESRD patients being diagnosed each year. The majority of these patients range from 45-64 years of age (40.9% of the class) or from 65-74 years of age (19.8% of the class). ESRD affects males (55% of the class) more than females (45% of the class); and afflicts Caucasians patents (60% of the class) more than twice as often as black/African-American patients (32% of the class). Lastly, the price for medically treating ESRD continues to rise; for example, the cost to the Federal government for the medical management of ESRD is currently 17.9 billion dollars annually.

### B. Hemodialysis

Currently, hemodialysis is the primary modality of therapy for patients with ESRD. A hemodialysis machine pumps blood from the patient, through a dialyzer, and then back into the patient. Hemodialysis therapy is thus an extracorporeal (i.e., outside the body) process which removes toxins and water from a patient's blood; and requires a constant flow of blood along one side of a semipermeable membrane with a cleansing solution, or dialysate, on the other. Diffusion and convection allow the dialysate to remove unwanted substances from the blood while adding back needed components. In this manner, the dialyzer removes the toxins and water from the blood by a membrane diffusion principle.

Hemodialysis is most often performed as an out patient procedure in approximately 3,600 approved centers in the U.S. In comparison, home dialysis is an option that is becoming ever less popular because of the need for a trained helper, large-sized dialysis equipment, and the very high costs. Typically, a patient with ESRD disease requires hemodialysis three times per week. Each session usually lasts for 3-6 hours depending on patient size, type of dialyzer employed and other medical factors.

### C. The Need for a Vascular Access

Removing blood from the body in order to filter the blood in the dialysis process requires a vascular access to the patient's blood system. A vascular access can be obtained in the short term via the use of percutaneous implanted catheters; but such short-term apparatus and methods ultimately must be replaced by long term procedures—which typically include surgically modifying the patient's own blood vessels to create an arteriovenous (“A-V”) fistula or surgically implanting a pre-formed prosthetic graft into the individual's blood vessels. In these long-term techniques, the vascular access site (such as the A-V fistula or prosthetic graft) lies entirely beneath the skin; and the skin and the internalized vascular access site must thus be punctured externally from outside the body using a syringe needle and blood tubing which is joined to the dialysis machine.

To be medically useful, the chosen mode of vascular access must remain patent (i.e., unblocked) and remain free from medical complications in order to enable dialysis to take place. The vascular access must also allow blood to flow to and return from the dialysis machine at a sufficiently high rate to permit dialysis to take place efficiently; and, desirably, it should allow the patient to carry on at least the semblance of a normal life.

However, the vascular access is widely called the “Achilles heel of dialysis” because of the markedly high morbidity and mortality among dialysis patients associated with complications of vascular access. Vascular access complications are believed to be the single greatest cause of morbidity; and, moreover, are believed to account for approximately one-fourth of all admissions and hospitalization days in the ESRD population.

Consequently, by virtue of the pathophysiology for the A-V access in humans, multiple revisions and replacement of the access itself is the rule in vascular access surgery. This combination of natural history failures, comorbidity and complications of therapy results in approximately 67,000 deaths attributed to ESRD in the U.S. alone. The medical and scientific literature evidences the severity of the problem. Merely illustrative of such medical and scientific printed publications are the following: Sidawy et al., “Seminars in Vascular Surgery”, AV Hemodialysis Access and its Management, Vol 17, No. 1., March 2004; Gibson et al, “Vascular access survival and incidence of revisions: A comparison of prosthetic grafts, simple autogenous fistulas, and venous transposition fistulas from the U.S. Renal Data System Dialysis Morbidity and Mortality Study”, J Vasc Surg 34:694-700 (2001); The Vascular Access Work Group, “NFK-DOQI clinical practice guidelines for vascular access”, Am J Kidney Dis 37 (suppl. 1):s137-s181 (2001); Puskas J. D. and J. P. Gertler, “Internal jugular to, axillaiy vein bypass for subclavian vein thrombosis in the setting of brachial a-v fistula”, J Vasc Surg 19:939-942 (1994); Fulks et al., “Jugular-axillary vein bypass for salvage of a-v access”, J Vasc Surg 9:169-171 (1980); Collins et al., “United States Renal Data System assessment of the impact of the National Kidney Foundation-Dialysis Outcomes Quality Initiative guidelines”,. Am J Kidney Dis 39:784-795 (2002); Kalrnan et al., “A practical approach to vascular access for hemodialysis and predictors of success”, J Vasc Surg 30:727-733 (2004); Palder et al., “Vascular access for hemodialysis: Patency rates and results of revision”, Ann Surg 202:235-239 (1985); Scher et al., “Alternative graft materials for hemodialysis access”, Sem Vasc Surg 17(1): 19-24 (2004); and Schuman et al., “Reinforced versus nonreinforced ptfe grafts for hemodialysis access”, Am J Surg 173:407-410 (1997).

### D. The Conventionally Known Means for Providing a Vascular Access

The need for vascular access in patients with renal failure can be either temporary or permanent. Devices and methods are available today to establish temporary vascular access for time periods ranging from several hours to several weeks. In comparison, permanent access methods and devices allow vascular access to a patient's blood system which typically last for months to years in duration.

In good medical practice, a temporary vascular access is typically used to treat patients with acute renal failure; patients in chronic renal failure without an available mode of permanent access; peritoneal dialysis patients or transplant patients needing temporary hemodialysis; and patients requiring plasmapheresis or hemoperfusion. In contrast, permanent vascular access devices and methods are the requisite rule for patients suffering from end stage renal disease.

A listing of the historically known, major kinds of vascular access is given below:

| | | Year Of First |
|---|---|---|
| Device & Technique | Type | Introduction |
| 1. Scribner shunt | Temporary Access | 1959/1960 |
| 2. Percutaneous catheter assembly | Temporary Access | 1983 |
| 3. A-V (arterio-venous) fistula | Permanent Access | 1966 |
| 4. Polytetrafluoroethylene (PTFE) | Permanent Access | 1977 |
| graft | | |

The Scribner Shunt:

The Scribner shunt was the earliest developed breakthrough percutaneous device which allowed patients afflicted with chronic kidney disease to have a temporary vascular access and the ability to be treated with the relatively primitive hemodialysis machines already-existing at that time. The device is an externally located arteriovenous shunt, developed in 1960 by Quinton and Shribner; and consists of two hard plastic cylinders or vessel tips. One vessel tip is implanted into an extremity artery and the other into a nearby vein; and the opposite vessel tip ends are connected to pieces of silicone elastomer tubing. After implantation, the two silicone tubes are connected with each other to establish the external shunt [see for example: E. Larson, L. Lindbloom and K. B. Davis, Development of the Clinical Nephrology Practitioner, Mosby, St. Louis, 1982; J. T. Daugirdas and T. S. Ing, Handbook of Dialysis, 2^{nd} Ed., Little, Brown and Co., 1994].

The Scribner shunt suffered from major infection and clotting problems; and required extensive post-operative and long-term care of the shunt. For these reasons, the Scribner shunt is today largely obsolete and is no longer used for hemodialysis.

The Percutaneous Catheter Assembly:

The second temporary method of vascular access is a percutaneous venous cannula assembly which inserted into a major vein—such as the femoral, subclavian or jugular vein. These catheter assemblies are percutaneous, with one end lying external to the body and the other end typically dwelling internally within either the superior vena cava or the right atrium of the heart. The external portion of these catheter assemblies has connectors permitting attachment of blood sets leading to and from a hemodialysis machine.

Typically, a percutaneous catheter assembly is a venous cannula having a catheter element and a connector portion comprising an extracorporeal connector element. In usual practice, the assembly's extracorporeal connector element is disposed against the chest of the patient; and the distal end of the catheter element is passed into a pre-chosen internal vein; and then is passed down through the vein into the patient's superior vena cava. More particularly, the distal end of the catheter element is usually positioned within the patient's superior vena cava such that the mouth of the suction line, as well as the mouth of the return line, are both located between the patient's right atrium and the patient's left subclavia vein and right subclavia vein. The percutaneous venous cannula assembly is then left in this position relative to the body, ready and waiting to be used during an active dialysis session.

Manner of Use

When hemodialysis is to be performed on the patient, the assembly's extracorporeal connector element is appropriately connected to a dialysis machine,—i.e., the suction line is connected to the input port (the suction port) of the dialysis machine; and the return line is connected to the output port (the return port) of the dialysis machine. The dialysis machine is then activated—i.e., the dialysis machine's blood pump is turned on and the flow rate set. The dialysis machine will withdraw relatively “dirty” blood from the patient through the suction line and return relatively “clean” blood to the patient through the return line. In practice, it has generally been found desirable to separate the assembly's two mouths by a distance of about 2 inches or so in order to avoid such undesired blood recirculation.

Perspective Chances Over Time

Percutaneous catheter assemblies have been used in hemodialysis since the early 1960's but for many years have been considered to be only a “temporary” form of vascular access because of their concomitant major infection and stenosis problems. However, because they can be easily and quickly inserted, they were used when emergency vascular access was needed to permit hemodialysis. Nevertheless, for many years, the risk of potentially life-threatening infection complications was considered to be so great that the percutaneous catheter assemblies were withdrawn after each dialysis session and re-inserted when necessary to minimize the risk of infection.

Yet, despite this history, two important developments occurred in the 1980's that have led some nephrologists to consider using percutaneous catheter assemblies as a “permanent” form of vascular access. The most important of these developments was a 1983 paper reporting the insertion of percutaneous catheter assemblies into the jugular vein rather than the subclavian vein. Jugular vein insertion essentially eliminated the problem of subclavian vein stenosis associated with up to 50% of subclavian vein catheter insertions. Note that subclavian vein stenosis not only blocks blood flow, making it impossible to conduct hemodialysis; but also, catastrophically, can destroy all potential vascular access sites in one or both arms.

The second major development was the attachment of a dacron “cuff” to the assembly's catheter element, near the proximal end, under the skin, about an inch from the incision site where the assembly exits the body. This cuff permits tissue in-growth to occur, which fastens the catheter element to the tissue and thereby reduces movement of the percutaneous catheter assembly at the incision site as well as in the blood vessel. In addition, such tissue in-growth is believed by many medical practitioners to retard bacterial travel along the outer surface of the percutaneous catheter assembly, although it does not prevent it entirely. Yet, while numerous published reports suggest that the cuff has reduced the infection rate, clinical infections remain a major problem even with the use of cuffed percutaneous catheter assemblies.

Nevertheless, because of these developments, a series of papers published in the 1990's reported positively on the long term survival of percutaneous catheter assemblies—thereby permitting and openly encouraging their use as a “permanent” form of vascular access. In addition, a wide range and variety of catheter apparatus improvements and catheterization method innovations have been generated which intend that venous cannula assemblies be employed as “permanent” means of vascular access. Merely exemplifying some of the most recent of these apparatus improvements and method of use innovations are the following: U.S. Pat. No. 6,758,841 entitled “Percutaneous Access”; U.S. Pat. No. 6,758,836 entitled “Split Tip Dialysis Catheter”; U.S. Pat. No. 6,685,664 entitled “Method And Apparatus For Ultrafiltration Utilizing A Long Peripheral Access Venous Cannula For Blood Withdrawl”; and U.S. Pat. No. 6,620,118 entitled “Apparatus And Method For The Dialysis Of Blood”. Each of these issued patents as well as the publications cited internally within them are expressly incorporated by reference herein.

The A-V (Arterio-Venous) Fistula:

A major method of permanent vascular access currently in use is the A-V (arterio-venous) fistula. By definition, an A-V fistula is a naturally occurring linkage or a surgical construct connecting a major artery to a major vein subcutaneously. For hemodialysis purposes, an anatomically-sited and purposefully created surgical construction is the practical reality.

A primary arteriovenous fistula is a preferred and cost-effective long-term access for hemodialysis patients. Because an A-V fistula is an artificial direct connection between an adjacent artery and vein, the high blood flow from the artery through this direct connection causes the vein to become much larger and develop a thicker wall, much like an artery. In this manner, the A-V fistula thus provides a high blood-flow site for accessing the circulatory system and for performing hemodialysis.

Via this new arterio-venous blood flow connection, most blood will bypass the high flow resistance of the downstream capillary bed, thereby producing a dramatic increase in the blood flow rate through the fistula. Furthermore, although it is not medically feasible to repeatedly puncture an artery, formation of the fistula “arterializes” the vein. The arterialized vein can be punctured repeatedly, and the high blood flow permits high efficiency hemodialysis to occur.

Manner of Use

For each dialysis, two large-bore needles (normally 14-16 gauge) are inserted through the dialysis patient's skin and into the A-V fistula, one on the “arterial” end and the other on the “venous” end. When the tips of the needles are properly resting inside the access, a column of blood enters the end of tubing attached to each needle. Prior to beginning a dialysis treatment, a cap is removed from each tubing, thereby allowing blood to fill the tubing, and then a syringe of saline is injected through each tubing and needle. The two needles are then connected with rubber tubing to the inflow (arterial) and outflow (venous) lines of the dialysis machine, and dialysis is started.

The A-V fistula today is still considered to be the “gold standard” for vascular access. Because of its comparatively longer survival time and relatively lower level of major problems, it is the widely preferred choice of nephrologists. However, data from the 1997 U.S. Renal Data System Report indicates that only about 18% of all hemodialysis patients currently receive a primary A-V fistula; while about 50% of patients receive a PTFE graft (see below) and about 32% of patients receive a percutaneous catheter assembly at about two months time after starting hemodialysis therapy.

Recognized Problems

One of the main reasons that the A-V fistula is not widely used is that the surgically-created-V fistula must “mature”. Maturation occurs when high pressure and high blood flow from the connected artery expand the downstream system of veins to which it is surgically connected. Surgeons have found that successful A-V fistula maturation is not possible in most hemodialysis patients because of the greatly increasing number of diabetic and older patients who have cardiovascular disease, which prevents the maturation process. Another reason for the low rate of usage is that since surgeons have failed so often to achieve fistula maturation after performing the costly A-V fistula surgery, the surgeon often will no longer even try this technique for creating a vascular access.

Another reason that A-V fistulas are relatively seldom used is that, even when fistula surgery is successful, the maturation of the constructed fistula generally takes approximately one to three months time to achieve. Since about half of all prospective patients have an immediate and urgent need to start hemodialysis as quickly as possible, the patient often cannot wait for A-V fistula maturation to occur. Thus critical patients must undergo costly temporary procedures and use percutaneous catheter assemblies to enable dialysis to take place, while waiting for maturation to occur.

In addition, it is one of the unfortunate drawbacks of A-V fistula, even with careful physical examination and/or the use of doppler ultrasound or venography to identify suitable veins, that approximately 40-50% of patients do not have the vascular anatomy sufficient to create a primary A-V fistula. In addition, many dialysis veterans, for whom the use of an A-V fistula has previously failed, can no longer be considered as candidates for a primary A-V fistula.

Finally, it will be noted that a number of innovations and improvements in the making and use of A-V fistula have been proposed and technically developed. Merely exemplifying these developments are U.S. Pat. Nos. 6,669,709; 6,585,760; 6,398,764; 6,113,570; 5,830,224; and 4,822,341. Each of these issued patents, as well as their internally cited publications, are expressly incorporated by reference herein.

The Prosthetic Graft:

The typical prosthetic graft is a linear hollow cannula formed of a durable and biocompatible synthetic material. Currently, most surgeons consider polytetrafluoroethylene (hereinafter “PTFE”), a “TEFLON” type of material, to be the synthetic material of choice. Although the prosthetic graft is essentially structured to be a flexible linear tube, a varied range of differences and modifications in fibril length, wall thickness, external wraps, and ring supports, internal coatings in prosthesis size and shape have been developed; and the present commercial manufactures of PTFE hemodialysis grafts offer a variety of choices. See for example the variety of different PTFE graft structures which are commercially available and sold today—as listed by Table 1, page 21, in Scher L. A. and H. E. Katzman, “Alternative Graft Materials for Hemodialysis Access”, Sem Vasc Surg 17 (1):19-24 (March, 2004).

When subcutaneously implanted by the surgeon, the PTFE prosthetic graft is integrally joined (by distal and proximal anastomoses) to a pre-chosen artery and a nearby vein in the arm; and thereby serves as a fluid flow connection and blood carrying bypass structure, which subsequently can be punctured by dialysis needle sets for vascular access and hemodialysis. Given the fact that A-V fistulas are largely not possible, a subcutaneously implanted PTFE prosthetic graft is today the most common form of permanent vascular access for the overwhelming majority of hemodialysis patients—because, in spite of the some severe limitations and risks for the conventionally known PTFE prosthetic graft, there simply is no better alternative available for them to date.

The usual locations for the subcutaneous insertion and anastomosis of a conventional PTFE prosthetic graft are typically in the forearm and the upper arm, and surgeons commonly use a PTFE prosthetic graft in either a loop or straight configuration. As a consequence, the choice of arterial blood vessels available for an inflow of blood into the PTFE prosthetic graft include the radial artery at the wrist, the antecubital brachial artery, the proximal brachial artery, the axillary artery, and rarely, the femoral artery. Similarly, the choice of venous blood vessel typically available for an outflow of blood from the PTFE prosthetic graft include the median antecubital vein, the proximal and distal cephalic veins, the bassilic vein in the upper arm, the axillary vein, the jugular vein, and the femoral vein.

The Presently Existing Problems of PTFE Grafts

Despite these recent improvements and advances in prosthetic graft technology, the frequency of PTFE graft failure in-vivo remains very high. There are many reasons for failure of an implanted PTFE prosthetic graft, infection, thrombosis and aneurysm formation being among them. However, the most common cause of failure by far is neointimal hyperplasia—as exemplified by the hyperplasia occurring at the venous side of the access graft anastomosis in an implanted prosthetic graft.

As shown by the photomicrograph of Prior Art Fig. A herein, neointimal hyperplasis results in the narrowing or “stenosis” of the distal outflow portion of the prosthetic graft device, and ultimately causes thrombosis of the entire length of the prosthetic graft, thereby rendering it unusable for dialysis. Although the thrombus can theoretically be removed, the underlying cause cannot; and thus the patient enters a spiral phase of recurrent failure, hospitalization and surgery. Despite innumerable attempts of various kinds over the years to prevent this particular cause of graft thrombosis and secondary failure, there have been few substantive advances to date.

Clearly therefore, the major disadvantages of the implanted PTFE prosthetic grafts are stenosis (i.e., closing of the lumen) and thrombosis (i.e., clotting), both of which block the flow of blood. This dysfunction occurs in almost all graft patients several times during their lives; and, because it interferes with life-sustaining dialysis, must be corrected quickly. Presently used interventional procedures include angioplasty to open the stenosis and infusion of thrombolytic agents such as urokinase to dissolve the clots. Also, various clinical studies report that the mean time for the operational use of the PTFE graft progressively decreases after each such corrective procedure; and such progressive decreases continue until the operational time is so short that the surgeon has little choice except to replace the graft. It is particularly noted that the survival time of the conventional PTFE graft, including all repairs necessary to maintain its function, currently averages only about two years.

Medical interventions to maintain PTFE prosthetic grafts and to treat patient complications (infection, thrombosis and aneurysm formation) are also expensive. Furthermore, declotting of the prosthetic graft is required every nine months or so on average. Also, because only three anatomic sites exist in each human arm for the placement of the prosthetic graft, the current medical practice is to perform additional screening procedures in an attempt to extend the survival time of the graft. Although these additional procedures add cost and inconvenience, they have yet to improve significantly the mean time interval between interventional repairs, although they may in fact improve the prosthetic graft survival life as such.

Overview:

In short, there remains a long standing and well recognized need for substantive improvements in prosthetic graft constructs and the manner of their surgical implantation subcutaneously. Moreover, a major clinical imperative exists today to find a more effective means for avoiding stenosis and thrombosis in the implanted prosthetic grafts as well as to reduce the frequency of the interventional repairs. Accordingly, were such improvements to be developed, the innovation would be recognized and accepted by medical practitioners and surgeons alike as being an unexpected development which provides major benefits and unforeseen advantages for the hemodialysis patient.

### SUMMARY OF THE INVENTION

The present invention has multiple aspects. A first aspect provides a subject-customized prosthetic endograft suitable for the carrying of flowing blood and serviceable after surgical insertion as a durable vascular access for long-term hemodialysis in a particular subject afflicted with end stage renal disease, said subject-customized prosthetic endograft comprising:
a flexible, elongated hollow tube construct formed of at least one durable and biocompatible material and comprised of
(i) a hollow ribbed medial section having a predetermined length, external diameter size, tubular wall thickness, and internal lumen diameter, and whose tubular wall can be repeatedly penetrated on-demand by dialysis needles;
(ii) a hollow distal conduit arm having two open ends, one open end terminating as a discrete distal conduit end and the other open end being integrally joined to and in fluid flow communication with said ribbed medial section, said distal conduit arm being of predetermined external diameter size, tubular wall thickness, and internal lumen diameter, and having a subject-customized linear length which is to be custom-sized by a surgeon such that after in-vivo insertion of said sized distal conduit arm into a pre-chosen vein, said distal conduit end will float freely within the vein and anatomically lie adjacent to the cavo-atrial junction of the heart in the particular subject; and
(iii) a hollow proximal conduit arm having two open ends, one end terminating as a discrete proximal conduit end and the other end being integrally joined to and in fluid flow communication with said ribbed medial section, said proximal conduit arm being of predetermined external diameter size, tubular wall thickness, and internal lumen diameter, and having a subject-customized linear length which is to be custom-sized by the surgeon such that said sized proximal conduit arm can be subcutaneously positioned over its entire sized length within the upper limb of the particular subject in-vivo, and said proximal conduit end can be surgically joined to and anastomosed at a pre-selected anatomic site with a pre-chosen artery in the upper limb of the particular subject.

A second aspect of the invention provides a surgical prosthetic endograft insertion kit whose components are used by a surgeon to create a durable vascular access suitable for long-term hemodialysis in a particular subject afflicted with end stage renal disease, said surgical prosthetic endograft insertion kit comprising:
(a) a subject-customized prosthetic endograft suitable for the carrying of flowing blood, which is configured as a flexible, elongated hollow tube and is constructed of at least one durable and biocompatible material, said prosthetic graft article comprising
   (i) a hollow ribbed medial section having a predetermined length, external diameter size, tubular wall thickness, and internal lumen diameter, and whose tubular wall can be repeatedly penetrated on-demand by dialysis needles,
   (ii) a hollow distal conduit arm having two open ends, one end terminating as a discrete distal conduit end and the other end being integrally joined to and in fluid flow communication with said ribbed medial section, said distal conduit arm being of predetermined external diameter size, tubular wall thickness, and internal lumen diameter, and having a subject-customized linear length which is to be custom-sized by a surgeon such that after in-vivo insertion of said sized distal conduit arm into a pre-chosen vein in the particular subject, said distal conduit end will float freely within the vein and anatomically lie adjacent to the cavo-atrial junction of the heart in the particular subject,
   (iii) a hollow proximal conduit arm having two open ends, one end terminating as a discrete proximal conduit end and the other end being integrally joined to and in fluid flow communication with said ribbed medial section, said proximal conduit arm being of predetermined external diameter size, tubular wall thickness, and internal lumen diameter, and having a subject-customized linear length which is to be custom-sized by a surgeon such that said sized proximal conduit arm can be subcutaneously positioned over its entire sized length within the upper limb in a particular subject, and said proximal conduit end can be surgically joined to and anastomosed at a pre-selected anatomic site with a pre-chosen artery in the upper limb of the particular subject;
(b) a flexible vascular graft obturator formed of durable material and having pre-determined dimensions and configuration, said vascular graft obturator having a tapered conical distal end, a rounded proximal end, and a central lumen able to accommodate the passage of a cable therethrough, and a withdrawl cable whose overall length passes through said central lumen;
(c) a tunneling obturator system comprising
a peel-away tunneling sheath of determinable length and volume, and
a central, conical-ended tunneling tool which can be locked into said tunneling sheath on-demand; and
(d) Seldinger technique workpieces comprising
a Seldinger needle of specific gauge,
a dilator of known linear length and diameter which has a plurality of measurement markers over its length, and
a guide wire of specified thickness and length.

A third aspect of the invention provides a surgical method for creating a durable vascular access suitable for long-term hemodialysis in a particular subject afflicted with end stage renal disease, said surgical method comprising the steps of:
(α) creating a first insertion site at a pre-selected anatomic position in the neck/shoulder of the particular subject to percutaneously puncture a pre-chosen vein;
(β) preparing a subject-customized prosthetic endograft configured as a flexible, elongated hollow tube and constructed of at least one durable and biocompatible material, said prosthetic graft article comprising
   (i) a hollow ribbed medial section having a predetermined length, external diameter size, tubular wall thickness, and internal lumen diameter, and whose tubular wall can be repeatedly penetrated on-demand by dialysis needles,
   (ii) a hollow distal conduit arm having two open ends, one end terminating as a discrete distal conduit end and the other end being integrally joined to and in fluid flow communication with said ribbed medial section, said distal conduit arm being of predetermined external diameter size, tubular wall thickness, and internal lumen diameter, and having a subject-customized linear length which is custom-sized by the surgeon such that after in-vivo insertion of said sized distal conduit arm into a pre-chosen vein in the particular subject, said distal conduit end will float freely within the vein and anatomically lie adjacent to the cavo-atrial junction of the heart in the particular subject,
   (iii) a hollow proximal conduit arm having two open ends, one end terminating as a discrete proximal conduit end and the other end being integrally joined to and in fluid flow communication with said ribbed medial section, said proximal conduit arm being of predetermined external diameter size, tubular wall thickness, and internal lumen diameter, and having a subject-customized linear length which is custom-sized by the surgeon such that said sized proximal conduit arm can be subcutaneously positioned over its entire sized length within the upper limb in a particular subject, and said proximal conduit end can be surgically joined to and anastomosed at a pre-selected anatomic site with a pre-chosen artery in the upper limb of the particular subject;
(γ) percutaneously passing said custom-sized distal conduit arm of said prosthetic graft article through said insertion site into the internal lumen of the pre-chosen vein in the particular subject, whereby said custom-sized distal conduit arm comes to rest entirely within the lumen of the pre-chosen vein, and whereby said distal conduit end floats freely and anatomically lies within the pre-chosen vein adjacent to the cavo-atrial junction of the heart in the particular subject;
(δ) creating a second insertion site at a second pre-selected anatomic position in the upper limb of the particular subject to gain access to a pre-chosen artery in the upper limb of the particular subject;
(ε) mobilizing a segment of the accessed pre-chosen artery in the upper limb of the particular subject;
(ζ) surgically forming a subcutaneous tunnel and open passageway within the upper limb which extends upwardly from said second insertion site and terminates adjacent to the first insertion site in the neck/shoulder of the particular patient, said formed subcutaneous tunnel and open passageway being substantially parallel to the anatomic location of the pre-chosen artery within the upper limb;
(η) passing said proximal conduit arm of said prosthetic graft article into and through the length of said subcutaneous tunnel and open passageway such that said custom-sized proximal conduit end lies adjacent to said second insertion site on the upper limb of the particular patient;
(θ) introducing said ribbed medial section of said prosthetic graft article through said first insertion site such said ribbed medial section lies subcutaneously adjacent to said open passageway and subcutaneous tunnel; and
(ι) joining and anastomosing said custom-sized proximal conduit end to said mobilized segment of the pre-chosen artery in the upper limb of the particular subject; and

(κ) surgically closing said first and second insertion sites.

### BRIEF DESCRIPTION OF THE FIGURES

The present invention may be more easily understood and better appreciated when taken in conjunction with the accompanying Drawing, in which:

Prior Art Fig. A is a photomicrograph showing neointimal hyperplasis, a medical condition which results in the narrowing (or “stenosis”) of the distal outflow portion of a conventionally known PTFE graft;

FIG. 1 illustrates a preferred embodiment of the prosthetic endograft in the present invention;

FIG. 2 illustrates a preferred embodiment of the endograft obturator in the present invention;

FIG. 3 illustrates the endograft obturator of FIG. 2 in relationship to the the prosthetic endograft of FIG. 1;

FIGS. 4A and 4B illustrate a preferred embodiment of the tunneling obturator of the present invention;

FIGS. 5A and 5B illustrate a preferred embodiment of the tunneling sheath of the present invention;

FIG. 6 illustrates the tunneling obturator of FIGS. 4A and 4B in relationship to the the tunneling sheath of FIGS. 5A and 5B;

FIG. 7 illustrates a preferred embodiment of the complete surgical insertion kit of the present invention;

FIGS. 8A-8F illustrate the steps of the modified Seldinger technique;

FIG. 9 illustrates the anatomic positioning of the major veins existing within the human arm;

FIG. 10 illustrates the anatomic positioning of the major arteries existing within the human arm;

FIG. 11 illustrates the anatomic positioning of the major veins lying within the human body with respect to the heart;

FIG. 12 illustrates the insertion of a guide wire extended through the internal jugular vein into the right atrium of the human heart;

FIG. 13 illustrates the insertion of a venous dilator over a guide wire extended through the internal jugular vein into the right atrium of the human heart;

FIGS. 14A-14C illustrate the interlocking placement of the obturator into the endograft at their distal ends;

FIG. 15 illustrates the precise placement of the end of the distal conduit arm for the endograft at the cavo-atrial junction of the heart;

FIG. 16 illustrates the location of the subcutaneous tunnel passageway created in the upper arm;

FIG. 17 illustrates the placement of the obturator withdrawn strand within the subcutaneous tunnel passageway created in the upper arm of FIG. 16;

FIGS. 18A-18B illustrate the interlocking placement of the obturator into the endograft at their proximal ends; and

FIG. 19 illustrates the proper internal positioning of the endograft as a whole within the human body as a durable vascular access.

### DETAILED DESCRIPTION OF THE PRESENT INVENTION

The subject matter as a whole which is the present invention provides a prosthetic endograft article, a surgical insertion kit, and an surgical insertion methodology for creating a vascular access in-vivo. In addition, the present invention is able to prevent a primary cause of arterio-venous graft thrombosis; and provides a novel vascular access construction for successful long term use in maintenance hemodialysis.

The present invention employs a prosthetic endograft which is patient-customized by the surgeon as an endovascular component; and utilizes a unique surgical method for endovascular insertion of the prosthetic endograft in a manner which does not require a distal anastomosis of the endograft, thereby allowing the distal outflow end of the implanted article to remain unattached and freely floating within the internal lumen of a pre-chosen vein.

The present invention is therefore able to provide a range of unforeseen advantages and unexpected medical benefits for the patient suffering from end stage renal disease. Among the unique advantages and significant medical benefits are the following:

(i) The present invention uses an endovascular approach to create a suture-less venous connection between the prosthetic endograft and the venous blood circulation of the patient's body. By definition, the term “endovascular” as used herein means the application of devices and/or methods within the blood vessel itself, usually percutaneously, in order to manipulate the anatomy and pathology of the blood vessel itself. Accordingly, the term “endograft” as used herein identifies the unique prosthetic graft article provided by the present invention which is to be operative and functional after implantation in the patient's blood vessels and circulatory system in-vivo.

(ii) The present invention employs an adaptation and modification of the endovascular surgical procedure commonly known as the “elephant trunk” technique to insert a prosthetic graft article and join the article to a pre-chosen artery and vein. As a major consequence of using this modified surgical protocol, there is no anatomic anastomosis between the distal end of the prosthetic article and the venous blood circulation of the patient.

(iii) The absence of a distal anastomosis between the implanted prosthetic graft article and the venous circulation negates pathological flow dynamics at their point of common contact and juncture. This, in turn, will avoid and obviate the initiation and generation of neo-intimal hyperplasia at the distal end of the endovascular prosthetic article, the endograft—which is recognized as being the most prevalent cause incidence of vascular thrombosis. Accordingly, via this series of medical avoidances, the in-vivo occurrence of neo-intimal hyperplasia will be substantially eliminated and the incidence of vascular thrombosis will become markedly reduced.

(iv) The patency rates of the implanted endograft will be significantly greater than ever before, thereby reducing the severity of problems encountered after insertion and markedly increasing the duration and effective life of the implanted prosthetic article for hemodialysis. As a direct consequence and outcome, the morbidity and mortality of the vascular access for performing maintenance hemodialysis will become substantially reduced.

### I. The Conceptual Origins of the Present Invention

Endovascular surgery encompasses those conventionally known medical procedures whereby a therapeutic device is placed intraluminally—i.e., within blood vessels—using minimally invasive or percutaneous techniques. However, endovascular surgery protocols have heretofore been used only to manage the pathology of the blood vessel itself, and have not ever before been used for the purposes of creating a durable vascular assess in-vivo. Thus, while the technology and process for using endovascular surgery is itself mature, this medical knowledge has always been severely restricted in its applications.

The subject matter as a whole which comprises the present invention is based upon a thorough understanding and utilization of conventional endovascular surgical protocols; but constitutes a major adaptation and substantive alteration of previously existing knowledge for an entirely new and different application; and employs a unique and meaningful modification of surgical technique for the express purpose of creating a vascular access. In particular, the present invention incorporates a combination of widely used open and percutaneous vascular surgery techniques with an endovascular component; and utilizes a newly structured prosthetic endograft and its associated implantation equipment and methodology. The structural components of the implanted prosthetic device as well as the manner of their surgical implantation are therefore completely original and unforeseen in their clinical application and result.

The now conventionally known technique—which has been adapted and substantively modified by the present invention—utilizes a concept popularized over a decade ago by Drs. Hans Borst and E Stanley Crawford known as the “elephant trunk” technique. Details of this original endovascular technique are given by Borst et al., “Extensive aortic replacement using ‘elephant trunk’ prosthesis”, Thorac Cardiovasc Surg 31:37-40 (1983); and Borst et al., “Treatment of aortic aneurysms by a new mutli-stage approach”, J Thorac Cardiovasc Surg 95:11-13 (1988).

In effect, these surgeons generated a set of surgical procedures for repairing complex thoraco-abdominal aneurysms. Specifically, they would invaginate a length of prosthetic graft material into the descending thoracic aorta as a temporary aid in order to stage a full and complete repair of a complex multisegment aortic aneurysm. Thus, these surgeons would first repair the proximal aortic segments; and, as a part of their initial procedure, they would implant a portion of the more distal graft material into the descending aorta without distal fixation. Then, at a subsequent stage, this previously implanted segment of the prosthetic graft, then floating freely in the descending intrathoracic aorta, would be incorporated into a completed aneurysm repair by employing a second vascular anastamosis (or several anastamoses) and another additional segment of vascular graft material.

In short, their multiple stage concept thus was, as a temporary measure and first stage surgical event, to implant intraluminally and initially leave a freely floating end of a prosthetic graft segment within the aorta without performing a distal vascular anastamosis. Then, as the requisite second stage event and followup surgical procedure, to introduce intraluminally and join a second additional segment of vascular graft material to the freely floating end of the previously implanted prosthetic graft segment as a distal vascular anastamosis and generate a complete aneurysm repair. This multiple stage surgical protocol has become the gold standard of treatment for repairing a complex aortic aneurysm.

Considerable medical literature has been published regarding the merits of the Borst and Stanley multiple stage surgical technique for repairing a complex aortic aneurysm. Merely illustrative and representative of these medical publications are the following: Kuki et al., “An alternative approach using long elephant trunk for extensive sortie aneurysm: elephant trunk anastomosis at the base of the inominate artery”, Circ 106 (12, Suppl 1): 1253-1258 (Sep. 24, 2002); Safi et al., “Staged repair of extensive aortic aneurysms: morbidity and mortality in the elephant trunk technique”, Circulation 104 (24):2938-2942 (Dec. 11, 2001); Zanetti, P. P., “Replacement of the entire thoracic aorta according to the reversed Elephant Trunk technique”, J Cardiovasc Surg 42 (3):397-4002 (January, 2001); and Keiffer et al., “Treatment of aortic arch dissection using the elephant trunk technique”, Ann Vasc Surg. 14 (6):612-619 (November, 2000).

### II. The Components of the Surgical Endograft Insertion Kit

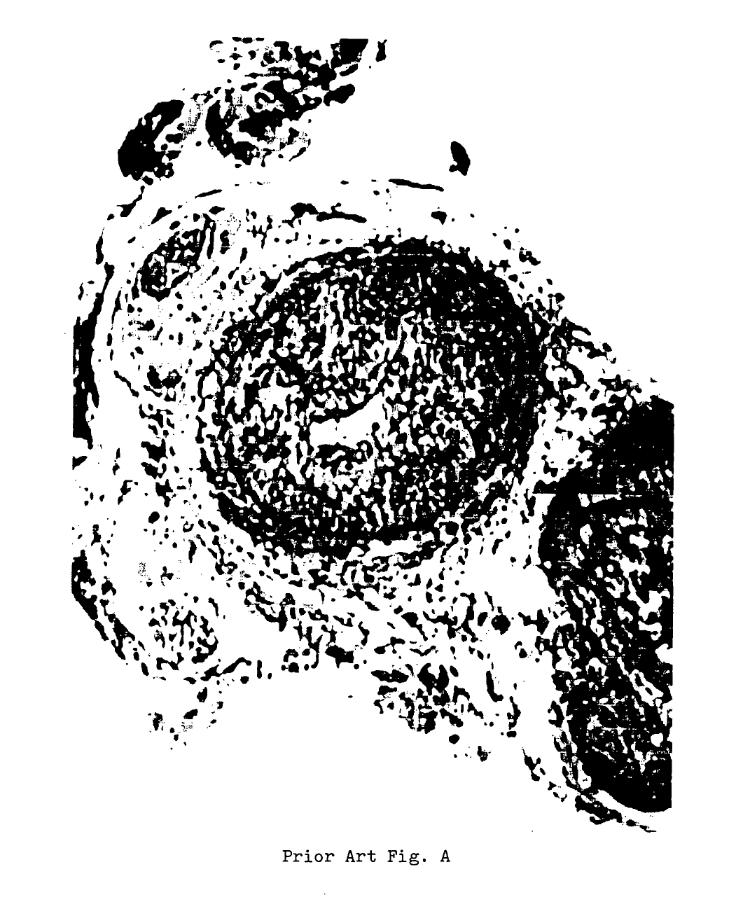

There are four article components which comprise the surgical insertion kit. These are: an endograft (the prosthetic graft article); an endograft obturator; a tunneling apparatus and system; and the Seldinger technique workpieces. Each of these components is described singly and as a complete insertion kit in detail hereinafter, ready for intended use by a surgeon; and these components are illustrated individually and collectively by FIGS. 1-7 respectively.

Component 1: The Prosthetic Graft Article (Endograft)

Desirably, the prosthetic endograft is a pref-formed, flexible and elongated hollow tube structure which is manufactured in a variety of different linear lengths, alternative exterior diameter sizes, varying wall thicknesses and differing inner lumen diameter sizes; and typically is composed of at least one durable and biocompatible material which may be entirely synthetic or be a derivative of living tissues. In addition, the durable material of the endograft structure offers a substantial flexibility for the inserted graft over the joints and anatomic bends in the body, and so prevents kinking of the endograft in-vivo.

In general, the pre-formed prosthetic endograft comprises three different structural component parts, as shown in detail by FIG. 1. These are: (I) the ribbed medial section; (ii) the distal conduit arm; and (iii) the proximal conduit arm.

(i) The ribbed medial section 20 of the endograft 10 illustrated by FIG. 1 is a hollow tube having two open ends 22. 24 as well as a predetermined length, external diameter size, tubular wall thickness, and internal lumen diameter. The circular tubular wall 26 of the ribbed medial section 20 is of a thickness and resilience which allows it to be repeatedly penetrated on-demand by dialysis needles whenever hemodialysis is to be performed. The ribs 28 are preferably disposed in a spiral pattern over the linear length of the medial section; and the ribs 28 serve as a structural reinforcement for the medial section over its intended long term of use.

(ii) The distal conduit arm 30 of the endograft 10 is an hollow tube having two open tubular ends 32, 34. One open end terminates as a discrete distal conduit end 32; while the other open end 34 is integrally joined to and lies in fluid flow communication with the open end 22 of the ribbed medial section 20. The distal conduit arm 30 is of predetermined external diameter size, tubular wall thickness, and internal lumen diameter. The distal conduit arm 30 also has an originally manufactured linear length which is to be shortened and custom-sized by a surgeon subsequently for the particular patient such that—after in-vivo insertion of the custom-sized distal conduit arm into a pre-chosen vein—the distal conduit end 32 will float freely within the internal lumen of the vein and anatomically lie adjacent to the cavo-atrial junction of the heart (but not actually within the atrium as such) within the particular subject.

(iii) The proximal conduit arm 40 of the endograft 10 is a hollow linear tube having two open tubular ends 42, 44. One open end 42 terminates as a discrete proximal conduit end, while the other open end 44 is integrally joined to and in fluid flow communication with the open end 24 of the ribbed medial section 20. The proximal conduit arm 40 is a tubular segment of predetermined external diameter size, tubular wall thickness, and internal lumen diameter. The proximal conduit arm 40 also has an originally manufactured linear length which is intended to be shortened and custom-sized subsequently by the surgeon such that the sized proximal conduit arm can be subcutaneously positioned over its entire sized length within the upper limb of the particular subject in-vivo, and the proximal conduit end can be surgically joined to and anastomosed at a pre-selected anatomic site with a pre-chosen artery in the upper limb of the particular subject.

### A Preferred Embodiment

In the preferred embodiment illustrated by FIG. 1, the prosthetic endograft is a tubular structure comprised of expanded PTFE, is about fifty five (55) cm in overall linear length, and is about six (6) mm in diameter. However, the total linear length of an endograft typically may vary from about 30-60 cm; and the exterior diameter of an endograft may vary in size from about 4-8 mm.

The preferred expanded-PTFE endograft has a spiral ribbed medial section which is preferably about fifteen to twenty (15-22) cm in length. It is integrally joined to and is in fluid flow communication with a distal conduit arm and a proximal conduit arm. The distal conduit arm of the endograft is a hollow tube, preferably about twelve to fifteen (12-15) cm in length; and terminates as a discrete distal (blood outflow) conduit end. Similarly, the proximal conduit arm of the endograft is also a hollow tube, preferably about fifteen to eighteen (15-18) cm in length; and terminates as a discrete proximal (blood inflow) conduit end.

It is very desirable that there be a series of radiographic markers disposed upon the exterior surface of the distal conduit arm at pre-measured distances and fixed intervals along its length up to the distal conduit end. These radiographic markers will typically be sub-millimeter sized titanium markings impregnated into the graft material itself, preferably at exactly one centimeter length distances. The markers will be visible both fluoroscopiclally and radiographically; be MRI (magnetic resonance imaging) compatible; and be used for measuring the exact distance and identifying the precise location of the distal conduit arm. In particular, these radiographic markers will provide an identifiable image of and visualization of the anatomic positioning for the distal conduit arm within the lumen of the pre-chosen vein; and permit accurate placement of the discrete distal conduit end such that it lies adjacent to the cavo-atrial junction of the heart (but not actually within the atrium as such) within the particular subject.

The Presently Existing Variety of PTFE Materials for Fabricating Endografts

A wide range and variety of different PTFE chemical formulations and compositions, methods of manufacture, and fabrication formats are commonly known and used today. Merely exemplifying the diversity of these PTFE materials and modes of fabrication are: The laminated self-sealing vascular access graft of U.S. Pat. No. 6,319,279; the PTFE vascular graft and method of manufacture described by U.S. Pat. No. 6,719,783; the dialysis graft system with self-sealing access ports disclosed by U.S. Pat. No. 6,261,257; and the self-sealing PTFE vascular graft and manufacturing methods recited by U.S. Pat. No. 6,428,571. In addition, a varied range of structural modifications differing in fibril length, wall thickness, external wraps, and ring supports, internal coatings in prosthesis size and shape are presently known. See for example U.S. Pat. Nos. 4,082,893; 4,177,334; 4,250,138; 4,304,010; 4,385,093; 4,478,898; 4,482,516; 4,743,480; 4,816,338; 4,478,898; 4,619,641; and 5,192,310. Accordingly, the text of each of these issued patents, as well as their internally cited publications, is expressly incorporated by reference herein.

Presently Available Alternative Biocompatible Materials for Endografts

The biocompatible composition comprising the material substance of the prosthetic graft article, however, is not intended to be confined or to be limited to the use of PTFE (in any of its conventionally known chemical formulations). To the contrary, a range and variety of different and alternative graft materials are presently available. Among these alternative materials are: “DACRON” or polyethylene terephthalate fibers and fabrics which were used as one of the original materials for prosthetic grafts (U.S. Pat. No. 2,465,319 assigned to Dupont Chemical Corp.); multi-layered and self-sealing polyurethane (manufactured by Thoratec, Pleasanton, Calif.); bioartificial matter derived from mesenteric vein (Hancock Jaffee Laboratories inc., Irvine, Calif.); and a cryopreserved allograft material in which cellular elements have been removed using antigen reduction technology (CryoLife Inc., Kennesaw, Ga.). Details and important considerations about these different and alternative graft compositions are described in Glickman, M. H., J Vasc Surg 34:45-472 (2001); Matsuura et al., Ann Vasc Surg 14:50-55 (2003); Bolton et al., J Vasc Surg 36:464-468 (2002); and Scher, L. A. and H. E. Katzman, Sem Vasc Surg 17 (1):19-24 (March, 2004).

Component 2: The Endograft Obturator.

The endograft obturator is a structure used to engage and carry the the prosthetic endograft towards its intended anatomic location and position. The obturator is preferably composed of a flexible and durable material; is short, about fifteen cm in length; and is typically configured to have a blunt proximal end and a tapered conical distal end. Also, within the solid body of the obturator, there is a central lumen of sufficient diameter to allow the passage therethrough of a 0.038 guide wire and a 100 cm flexible wire cable; and these are located intraluminally and will emerge from the blunt proximal end of the obturator to facilitate withdrawal of the obtrurator after the prosthetic graft article lies in the intended anatomic site.

### A Preferred Embodiment

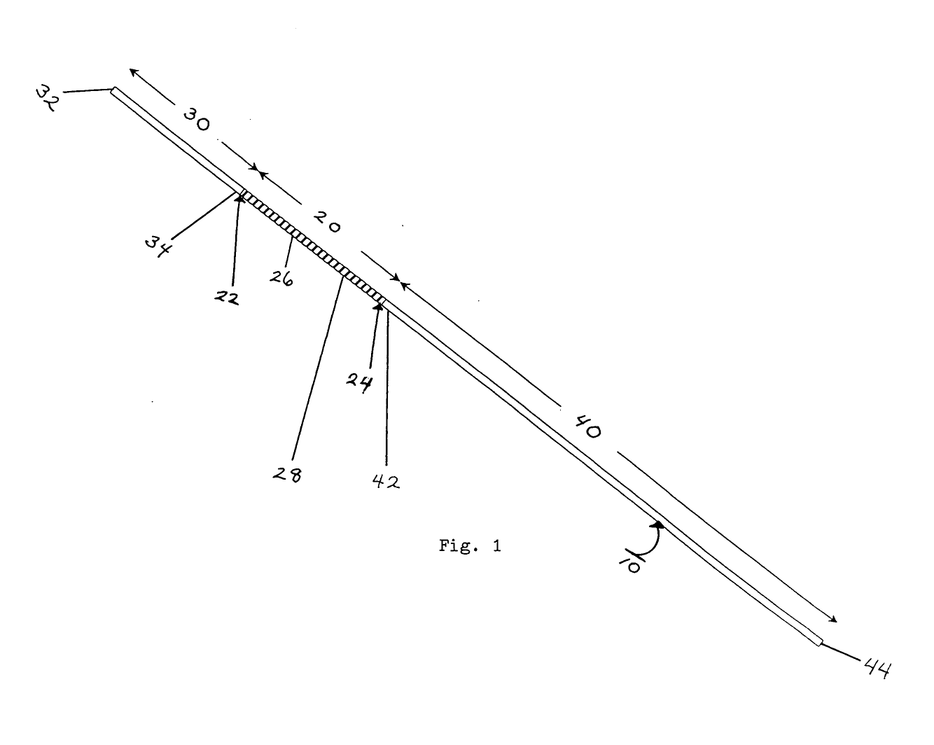

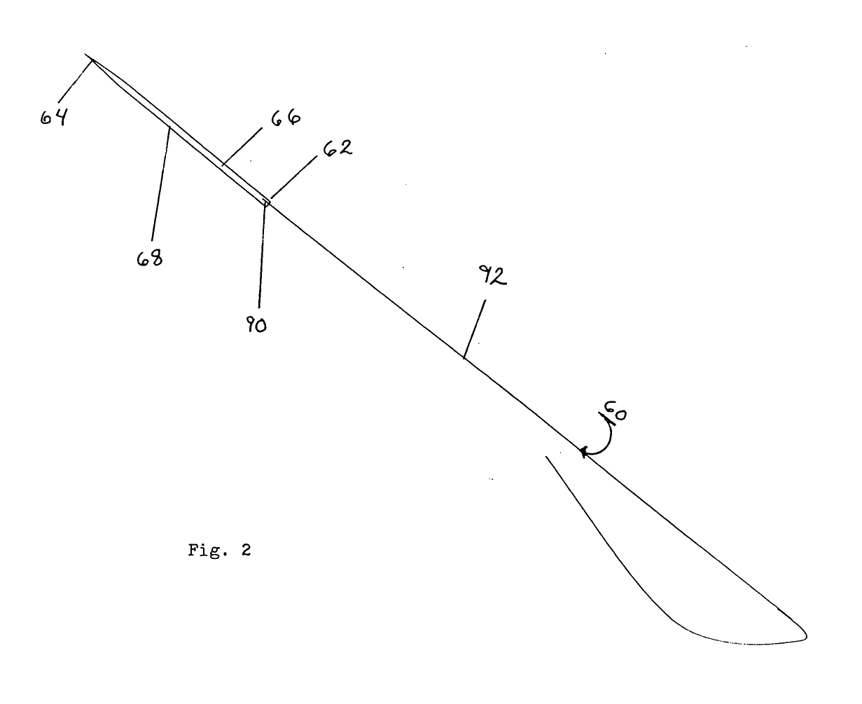

The endograft obturator is shown in isolation as a preferred embodiment by FIG. 2, and is shown in relationship to the endograft 10 by FIG. 3.

In the preferred embodiment of FIG. 2, the obturator 60 is desirably composed of a flexible and durable plastic material such as polyethylene or polystyrene. The obturator 60 will typically be about 15 cm. in length; and present a blunt proximal end 62 as well as a tapered conical distal end 64. Within the configured solid body 68 of the obturator 60, there is a central lumen 66, usually called the “obturator stylet guide”. This central lumen 66 will be of sufficient diameter size to allow the passage therethrough of a 0.038 cm guide wire 80, which typically is of a length varying from about 100-280 cm.

In addition, there are typically four individual withdrawl cables 90 securely imbedded within the material substance of the obturator 60; and these individual withdrawl cables 90 are typically attached at the 12:00, 3:00, 6:00 and 9:00 clock positions. All four individual withdrawl cables 90 are usually formed of the same material as the obturator 60, but each withdrawl cable 90 is only approximately 0.038 cm in thickness. Immediately upon exiting the proximal end 62, the four withdrawl cables are intertwined around one-another (during manufacture) such that they form, for all practical purposes, a single integrated strand 92.

Upon exiting the body of the obturator 60, the single integrated withdrawl strand 92 will present a linear length of approximately 65 cm. The intended purpose and function of the single withdrawl strand 92 is to facilitate the withdrawl of the obturator 60 (and the endograft assembly as a whole) through a tunnel sheath in-vivo—such that the endograft comes to lie in its intended anatomic location proximally, preferable at or adjacent to the brachial artery or axillary artery site, in a manner ready for anastamosis subsequently.

Additionally, once the obturator 60 has properly anatomically positioned the prosthetic endograft in place within the venous system at the level of the atrio-caval junction, the obturator 60 will itself be pulled back (by the surgeon) through the remaining portions of the endograft until it reaches the proximal conduit end. There, at precisely the proximal conduit end of the endograft, will be a circumferential intraluminal ring which will (I) stop the further egress of the obturator; and (ii) lock the obturator in place so that it can go no further. This locked connection of the endograft 10 and obturator 60 will eventually allow this locked-system to be pulled through the tunnel sheath in-vivo and come to lie in its final proximal location in the upper arm, ready for surgical juncture and fluid flow connection to the artery of choice.

Component 3: The Tunneler Apparatus & the Tunneling System

The complete insertion kit of the present invention also provides an apparatus for tunneling a passageway subcutaneously within the soft tissues in the upper arm of a living human patient. Preferably, the apparatus comprises a peel-away tunneling sheath and a central conical ended obturator which can be locked into the tunneling sheath.

Conventionally Available Tunneling Apparatus and Systems

It will be recognized and appreciated that the surgical implantation of the endograft is to be made subcutaneously within the soft tissues beneath the skin of the patient; and that when the in-vivo surgical procedure is completed, there are no structural elements or portions of the prosthetic endograft that are visible or exposed on the exterior surface of the patient's skin.

To achieve the desired implantation, a tunnel passageway must be created subcutaneously in-vivo; and a variety of surgical tunneler methods and tunneling devices are presently known and commercially available for this purpose. Merely illustrating and representative of the currently available tunneling devices and tunneling methods are those described by U.S. Pat. Nos. 5,306,240; 4,832,687; 4,574,806; and 4,453,928. The text of each of these issued patents, as well as their internally cited publications, is expressly incorporated by reference herein.

A Preferred Tunneling Apparatus and System

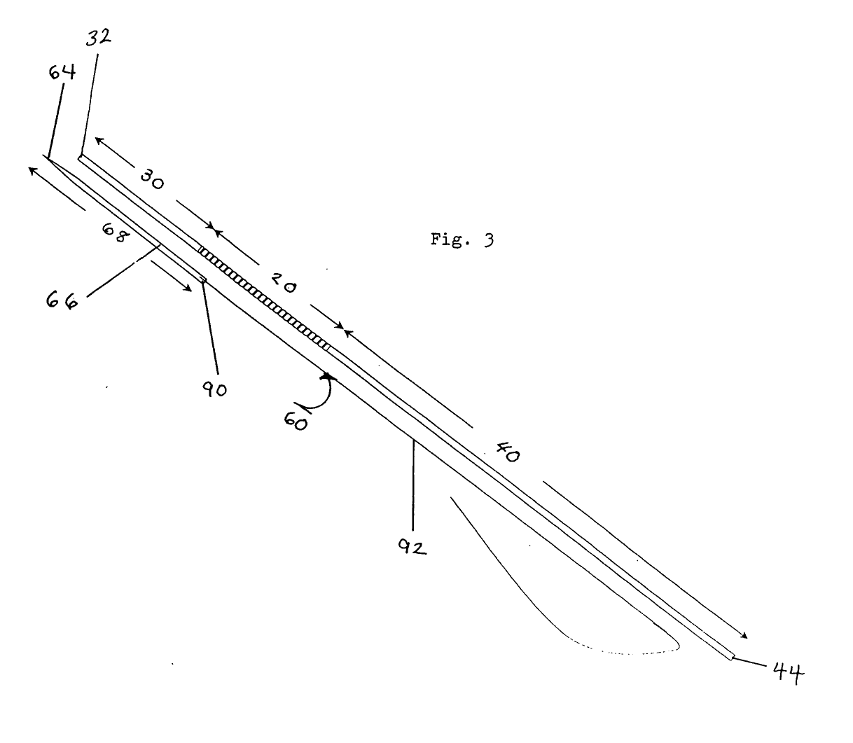

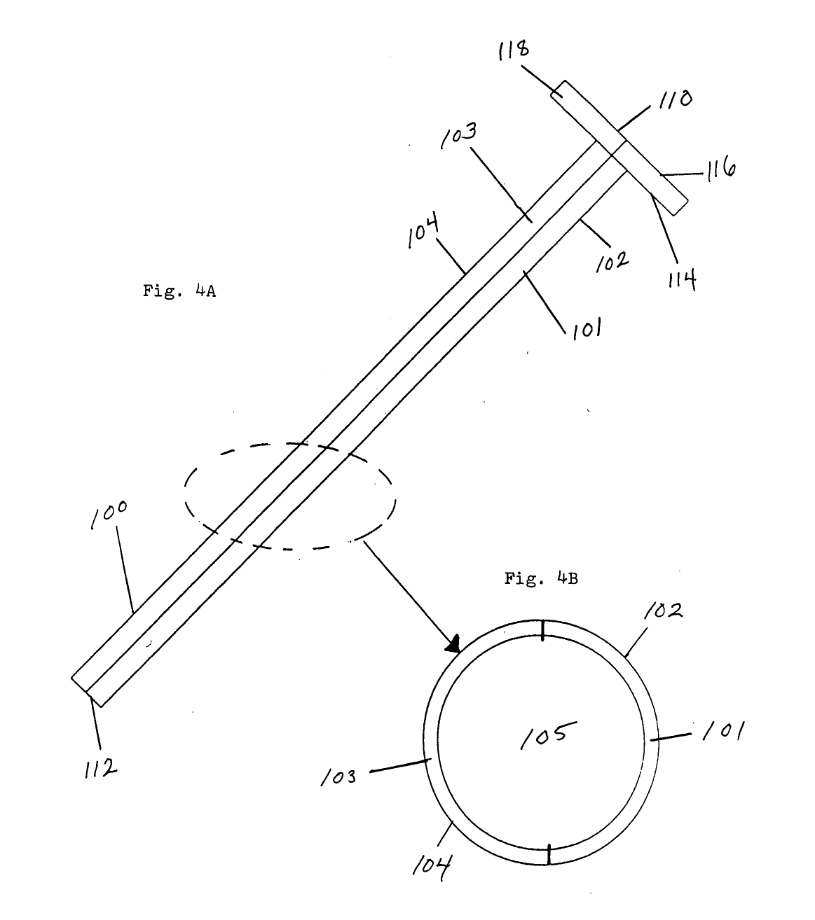

A preferred tunneling apparatus is a two-part system comprised of a tunnel sheath and a tunnel obturator. Both parts will be made of a material like polyethylene or polyurethane or polystyrene; and each part has sufficient structural rigidity to be passed into and through the subcutaneous tissue of a patient in-vivo in order that a tunnel passageway may be made in-situ. A preferred tunneling apparatus and system is illustrated by FIGS. 4-6 respectively.

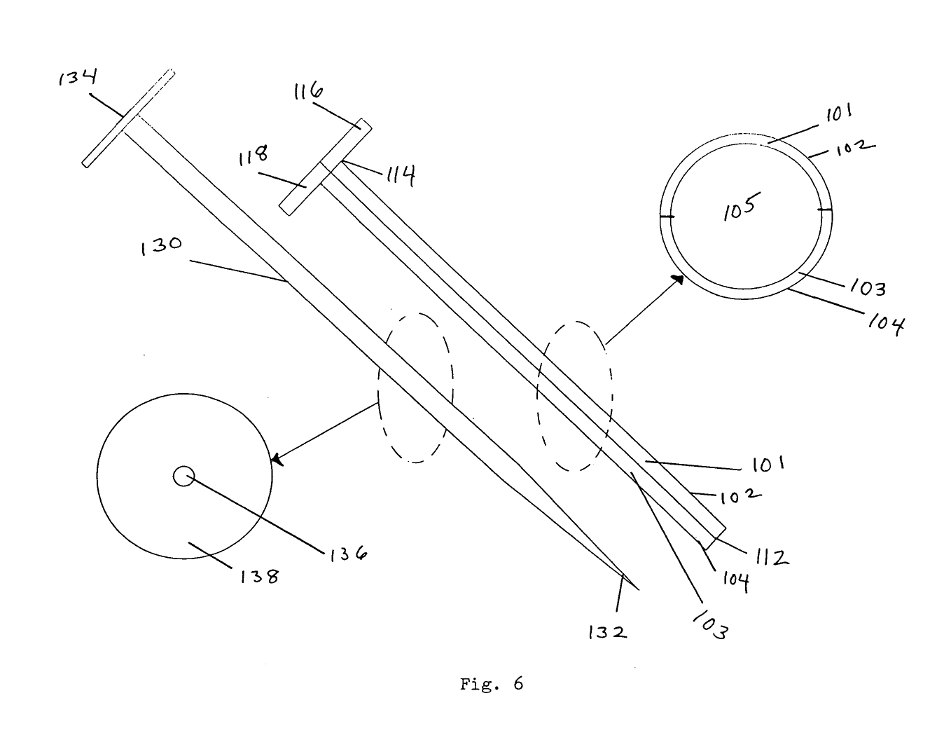

As shown by FIGS. 4A, 4B and 6 respectively, the tunnel sheath 100 will typically be approximately 25 cm in length; be hollow; and be formed of two symmetrical halves 101, 103. These two symmetrical halves 101, 103 are fused together to produce a single lumen 105 of predetermined spatial volume within the fused sheath.

The fused sheath 100 has a proximal end 110 and a distal end 112. At the proximal end 110 of the sheath 100 is a “T”-shaped flange 114, approximately 3 cm in length; and positioned such that about a 1.5 cm sized first arm 116 of the flange 114 is connected to a first side 102 of the sheath. There is also about a 1.5 cm sized second arm 118, which is connected to a second side 104 of the sheath. When the two arms 116, 118 are fused together, the flange 114 will appear to have a “T”-shaped overall configuration.

The fused sheath 100 is structured such that when the two individual arms 116, 118 of the “T” shaped fused flange 114 are grasped and pulled apart in opposite directions, the sheath will separate, or “peel apart”—beginning at the proximal end 106 and progressing to the distal end 108—such that at the end of the manipulation there will be two symmetrical and separate sheath halves 101, 103. The overall girth and diameter size of the fused sheath 100 may be varied; and typically will be in the range of about 6-10 mm in diameter size, so as to provide a spatial volume which will accommodate a prosthetic endograft of a particular volumetric size. It is expected, however, that the fused sheath 100 will be manufactured in a range of differing diameter sizes so that an appropriate sheath size can be selected in advance by the surgeon.

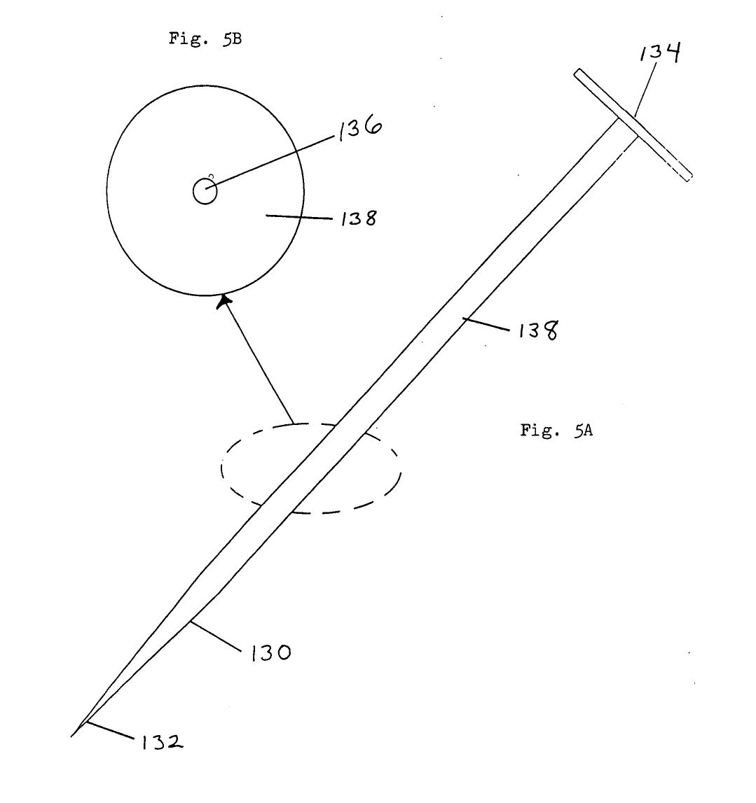

Also as shown in FIGS. 5A, 5B and 6 respectively, the tunnel obturator 130 is typically approximately 30 cm in length, has a distal conical tip 132, and presents a proximal end 134 in a “T”-shaped configuration. The tunnel obturator 130 has a solid body 138 and a small sized central lumen 136 which can accommodate a 0.038 cm guidewire therethrough; and preferably is composed of the same material as the tunnel sheath 100. The external diameter (except for the conical distal end) of the tunnel obturator 130 will be such that the obturator will fit within the internal spatial volume of the tunnel sheath 100 snugly and will be able to be withdrawn at will from the internal spatial volume of the tunnel sheath 100 without difficulty.

Component 4: The Seldinger Technique Workpieces

The Seldinger technique workpieces comprise a grouping which will typically include at least one thin-walled puncture needle 160 (preferably 18-22 gauge); a radiopaque vein dilator 170 (preferably 20-25 cm in linear length and typically of 5-6 French diameter size) which has a series of radiopaque (typically 1 cm sized) markers over its linear length; and at least one flexible guide wire 180 (preferably 0.038 inch thick and 100 cm in length). These items as a grouping are illustrated as individual component parts present within the complete insertion kit 200, as shown by FIG. 7.

The Modified Seldinger Technique:

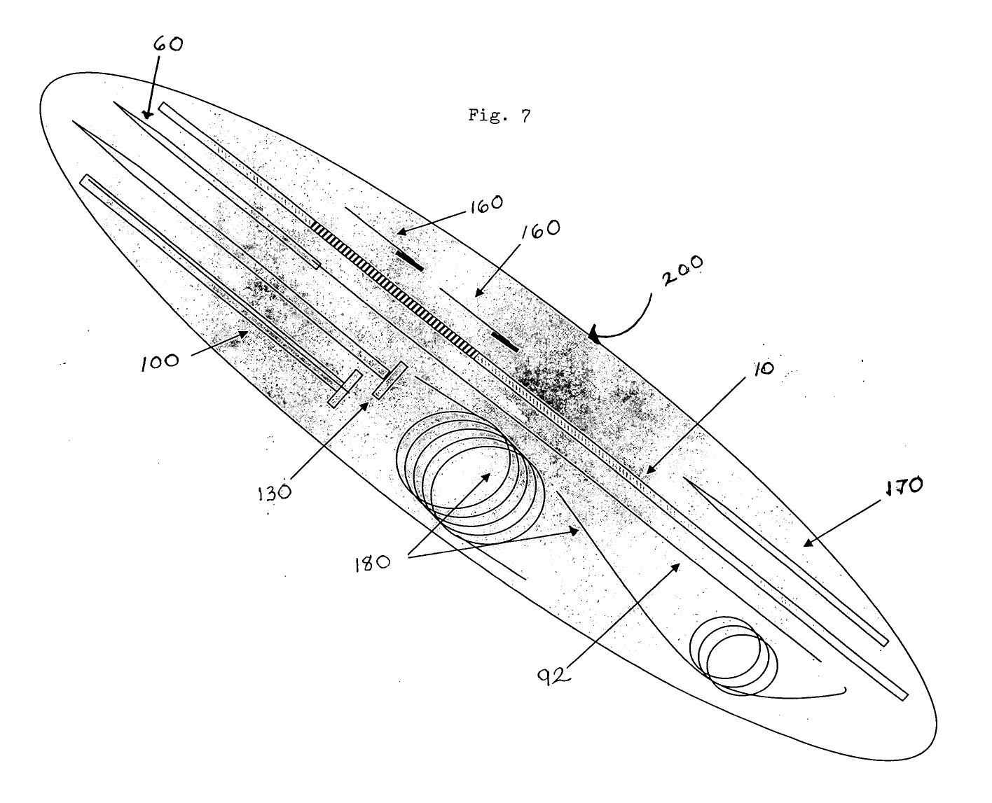

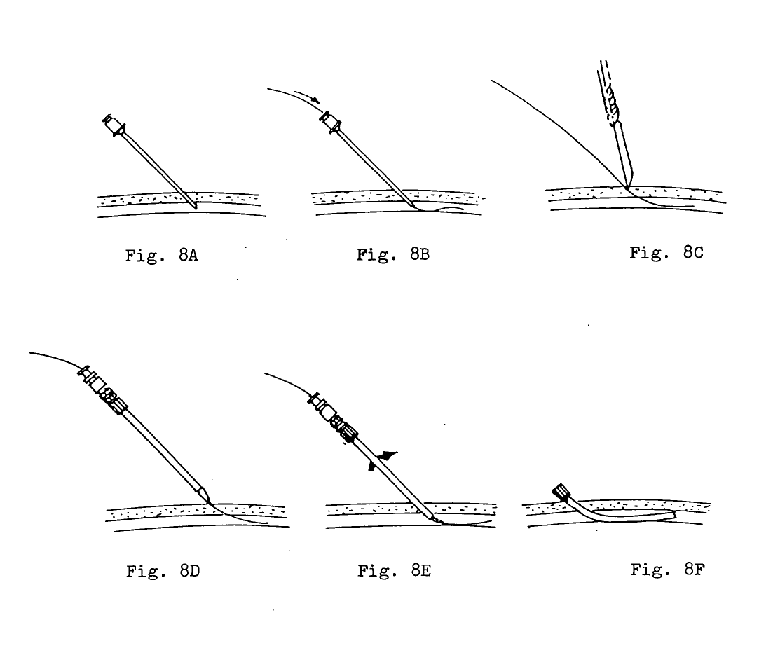

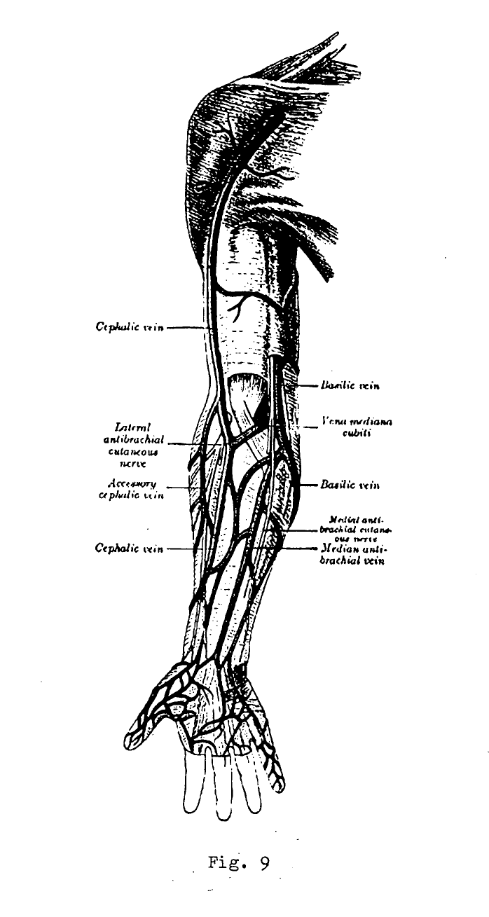

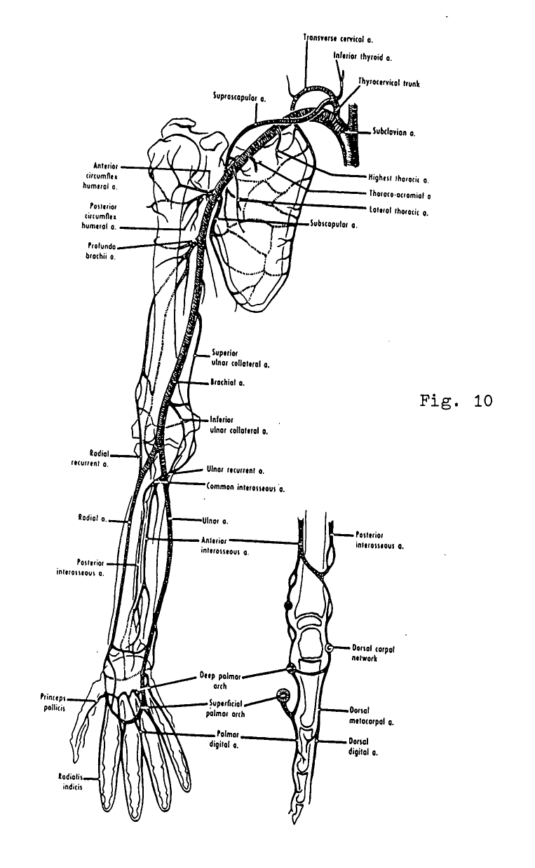

The percutaneous use of these workpieces is illustrated by the modified Seldinger technique which is shown by FIGS. 8A-8F respectively.

FIG. 8A shows a blood vessel being punctured with a small gauge needle, which has been percutaneously introduced through the epidermis and dermis by the surgeon. Once vigorous blood return occurs, a flexible guidewire is placed into the blood vessel via the bore of the needle as shown by FIG. 8B. The needle is then removed from the blood vessel, but the guidewire is left in place. Then the hole in the skin around the guidewire is enlarged with a scalpel as shown by FIG. 8C. Subsequently, a sheath and a dilator is placed over the guidewire as shown by FIG. 8D. Thereafter, the sheath and dilator is advanced over the guidewire and directly into the blood vessel as shown by FIG. 8E. Finally, the dilator and guidewire is removed while the sheath remains in the blood vessel, as illustrated by FIG. 8F. The prosthetic endograft is then inserted through the sheath and fed through the lumen of the blood vessel to reach the desired anatomic location.

### III. Anatomic Considerations

Clearly, the surgeon has a choice of which vein and which artery shall be employed and to be connected for blood carrying purposes via the prosthetic graft article and surgical methodology of the present invention. While somewhat limited in his selection of suitable blood vessels by the anatomy of the human body, the surgeon nevertheless has considerable leeway in choosing to employ one particular vein and one particular artery in combination, as is shown by FIGS. 9 and 10 respectively.

For these reasons, merely to illustrate the most typical and frequently used combinations of veins and arteries is the non-exhaustive and representative preferred listing of Table 1 below.

TABLE 1
Desirable Combinations

| Choice of vein | Choice of artery |
|---|---|
| Jugular vein | Brachial artery |
| Axillary vein | Axillary artery |
| Femoral vein | Femoral artery |
| Subclavian vein | Subclavian artery |

### IV. The Surgical Method of the Present Invention

### A. An Overview of the Surgical Insertion Methodology

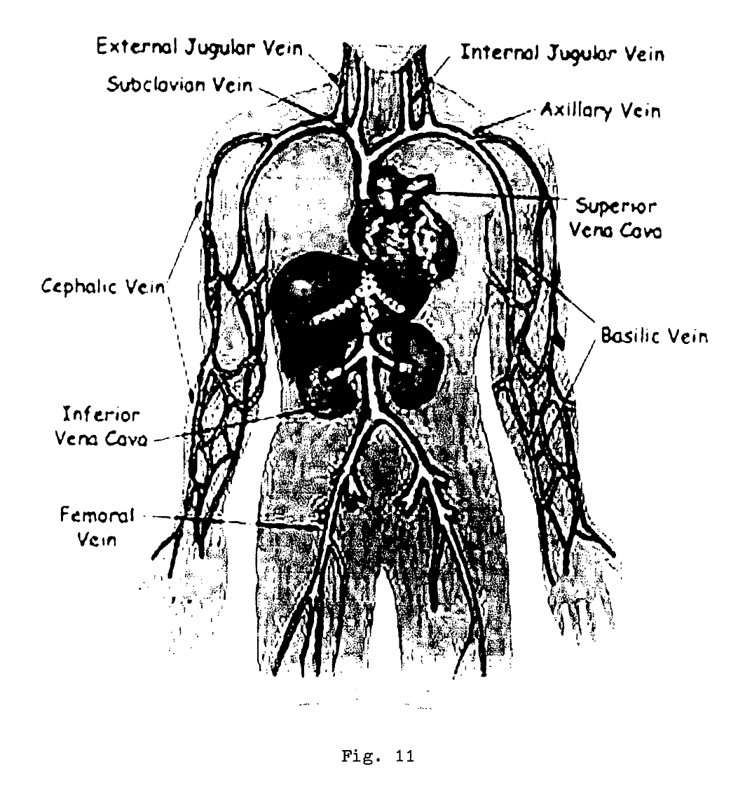

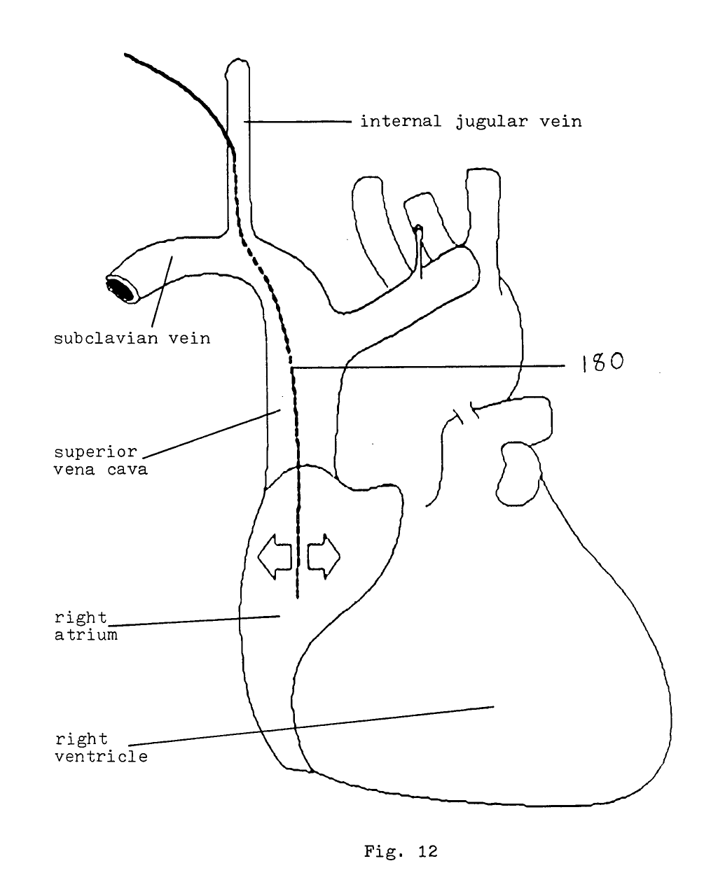

A summary description of the most preferred surgical insertion method—which will be recited again in greater detail hereinafter and is illustrated by FIGS. 11-19 respectively—is the following: A prosthetic endograft is inserted percutaneously into the right jugular vein and then is passed under fluoroscopic guidance to the level of the cavo-atrial junction of the right atrium. The prosthetic endograft is then subcutaneously tunneled into the arm of the patient from its insertion sue in the right lower neck area; is passed down over the shoulder; and then exits over and into a segment of the right brachial artery for anastamosis. This anastamosis site can vary in anatomic location from just above the elbow crease in the medial bicipital groove, to just below the right axilla, in the proximal bicipital groove. At the selected inflow site, a small incision is made in the skin, the brachial artery is isolated and the proximal anastamosis of the inflow limb of the graft is completed using standard vascular surgical techniques.

The described surgical methodology and insertion technique therefore provides not less than four major benefits and unique advantages:

1. The methodology uses an endovascular approach to create a suture-less venous connection between the endograft and the venous circulation. Thus, a rapid, hemostatic, maximally patent connection is created with this technique. In this minimally invasive way, and by avoiding the standard open surgical techniques, an improved durable connection is made which markedly reduces the risks of potential infection and healing difficulties resulting from a standard conventional surgical procedure.

2. Neointimal hyperplasia, as shown in the radiograph, occurs at the distal anastamosis outflow end of the endograft. By employing a modified “elephant trunk” technique—and because there is no vascular anastamosis between the graft and the outflow venous vessel—the negative pathologic flow dynamics (leading to vascular neointimal hyperplasia, subsequent graft thrombosis, and failure) will be obviated completely. As a consequence, the subsequent long-term patency of these endografts will be significantly greater, and markedly prolong the effective durability and safety of vascular access procedures.

3. In addition, because the venous end of the endograft is at the level of the right atrium, potentially higher blood flow rates will be obtained which are not limited by smaller sized veins. This markedly reduces the actual dialysis time for the patient and improves the efficiency of the dialysis process itself.

4. Finally, by utilizing the open and free-floating elephant trunk venous connection, if and only if thrombosis of the endograft does occur for other reasons than neointimal hyperplasia, subsequent de-clotting (or thrombectomy) will be more easily facilitated and completed because of the flow dynamics of such a vascular anastamosis.

### B. A Detailed Recitation of the Surgical Insertion Method

For purposes of providing the user with a clear comprehension and better appreciation of the present invention as a whole, a detailed anatomic description of a preferred surgical method and technique for the insertion of a prosthetic endograft is stated below.

It will be expressly understood, however, that the details of the surgical technique described herein, as well as the choices of anatomic location and of specific vein and artery employed, are no more than a preferred embodiment and single example of the method; and as such, are presented solely as one desirable set of representative and illustrative choices for the surgical methodology as a whole. For these reasons, the intended user of the present invention will recognize and acknowledge that a wide range of alternative anatomic locations for insertion is available to the surgeon; and that a substantial variety and range of choice for a particular vein and artery to be used in combination exist (as shown by the listing of Table 1).

(i) Anatomic Considerations:

A general anatomic positioning of the heart and the venous circulation is shown by FIG. 11. The user is presumed to be both cognizant and familiar with the different anatomic locations and positional relationships among the different major veins in the human blood circulatory system and the heart itself. FIG. 11 is therefore merely a convenient guide and reference model embodying conventional human anatomy and medical knowledge.

(ii) The Venous Implantation Component of the Surgical Procedure:

1. Using the conventionally known Seldinger technique (illustrated herein by FIGS. 8A-8F), at a first incision site 300 a needle puncture of the right internal jugular vein is performed, utilizing either a standard anterior or posterior supraclavicular approach. A 0.038 inch flexible guide wire 180 is then passed through the puncture needle 160 and threaded under fluoroscopic control through the cavo-atrial junction and into the right atrium of the patient's heart. This is illustrated in part by FIG. 12.

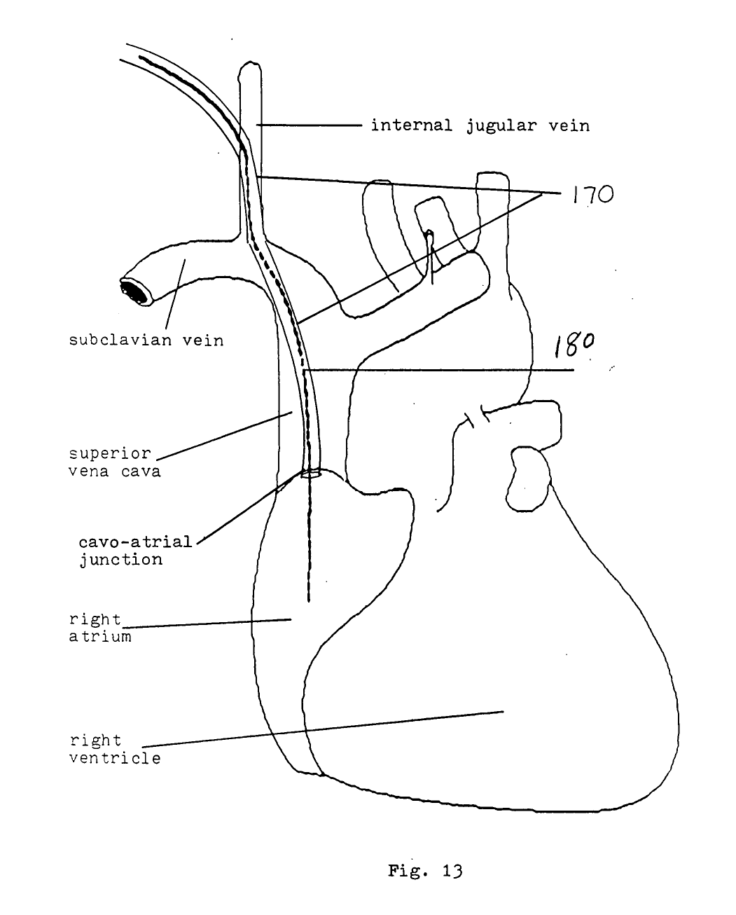

2. Removing the puncture needle 160 while securing the guide wire 180 in place, a 5 French angiographic dilator catheter 170 is then passed over the guide wire 180 to the level of the cavo-atrial junction of the patient's heart. This step is illustrated by FIG. 13.

3. Using the one centimeter radiopaque markings disposed on the dilating catheter 170, the linear distance from the jugular vein entry site to the cavo-atrial junction of the patient's heart is measured and confirmed using a limited contrast medium injection. This empirically measured linear distance serves as the subject-customized distal conduit length parameter.

4. Then, the surgeon carefully measures and cuts the endovascular distal conduit arm 30 of the prosthetic endograft 10 such that its (blood outflow) distal conduit end 32 extends the same measured linear distance from the junction of the ribbed medial portion 20 over the distal conduit arm. This will provide a patient-customized distal conduit arm length for the prosthetic article whose distal conduit end, after insertion, will lie properly in anatomic position adjacent to (but not actually within) the cavo-atrial junction of the patient's heart.

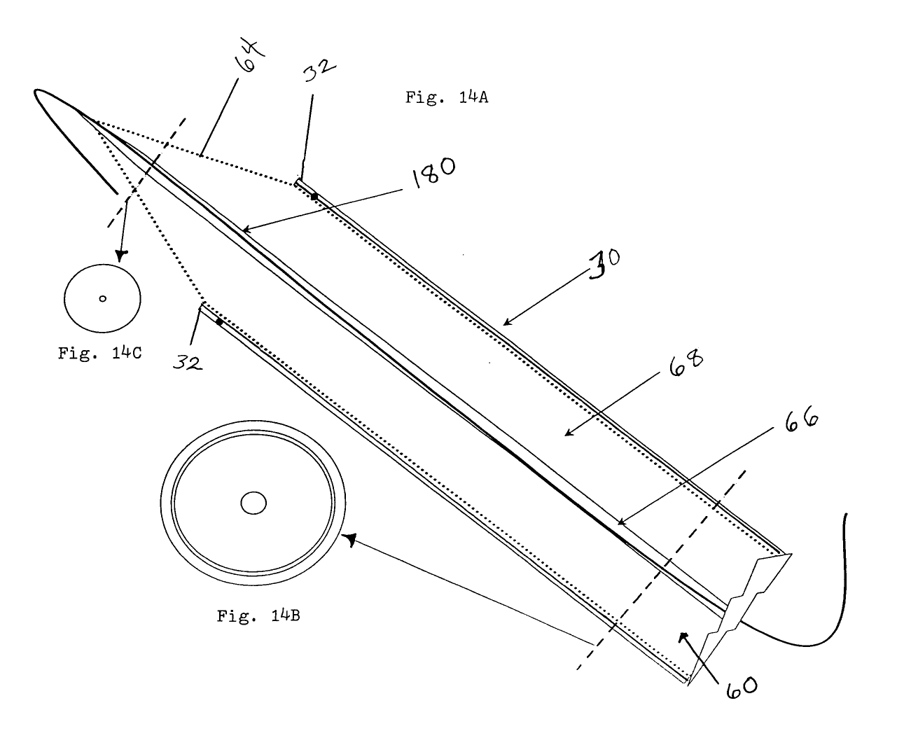

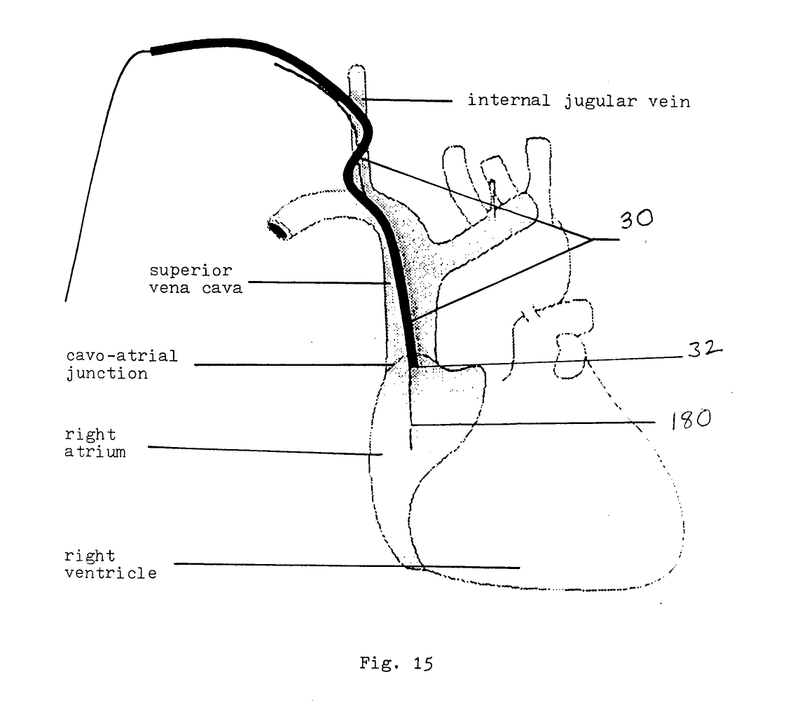

5. The surgeon inserts the tapered distal end 64 of the flexible obturator 60 into the distal conduit arm 30 of the prosthetic endograft 10; and then extends the obturator 60 until its tapered distal end 64 becomes exposed beyond the cut distal end 32 of the distal conduit arm 30 of the endograft 10. This is illustrated by FIGS. 14A, 14B, and 14C respectively.

6. Make a small transverse incision over the indwelling angiographic dilator catheter, thereby visualizing the entire internal tract of the dilator 170 and guide wire 180 lying within the catheter 160. Then, enlarge the original entry hole at the first incision site 300 over the jugular vein (the venotomy site) such that it will comfortably accommodate the distal conduit arm 30 and distal conduit (blood outflow) end 32 of the endograft 10.

7. Using the distal end radiographic markers of the endograft 10 as a guide, the obturator body 68 and custom-sized distal conduit arm 30 are advanced over the guide wire 180 until the distal conduit end 32 lies at the level of the cavo-atrial junction. This maneuver should bring the ribbed medial portion 20 of the endograft 10 into direct physical contact with the venotomy site in the jugular vein. This is illustrated in part by FIG. 15.

8. Perform a limited injection of contrast medium at the distal conduit end 32 to confirm the correct placement and anatomic location of the distal conduit end 32 and the obturator body 68 to be adjacent to the cavo-atrial junction. Presuming that the placement and anatomic location of the distal conduit arm 30 is correct, the custom-sized distal conduit end 32 is now freely floating (without any distal anastomosis as such) at the cavo-atrial junction of the patient's heart. Once proper positioning is confirmed, the venous implantation portion of the surgical methodology is effectively complete.

(iii) The Arterial Implantation Component of the Surgical Procedure:

9. Locate the brachial artery in the upper arm of the patient. Make a small secondary incision in the skin over the brachial artery at the level of the intended proximal (blood inflow) anastamosis, preferably at a second incision site 310 lying adjacent to the patient's elbow. Identify the brachial artery and surgically mobilize it such that an end-to-side anastamosis to the brachial artery can be easily accomplished.

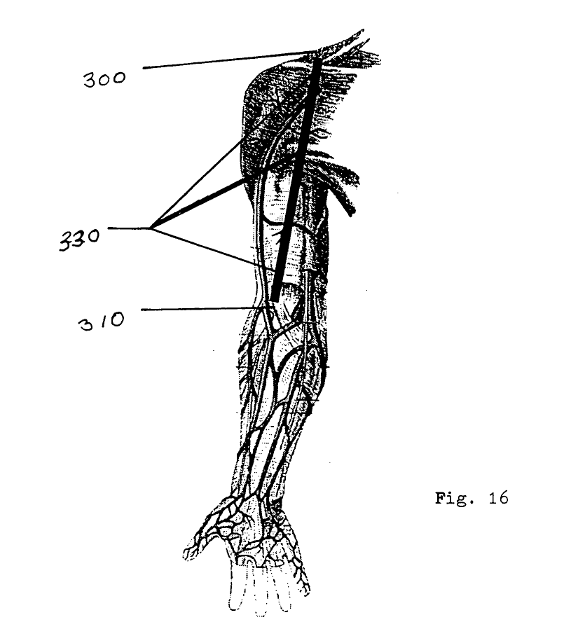

10. Through the secondary incision site 310 over the brachial artery, subcutaneously pass the tunneling device 130 and accompanying tunneling sheath 100 upwards (i.e., ascending towards the neck of the patient) within the soft tissues of the upper arm in a plane substantially parallel to the brachial artery and the pathway previously determined to optimize hemodialysis access. This effort will result in the creation of a subcutaneous tunnel and passageway 330 which lies and extends substantially parallel to the brachial artery within the upper arm of the patient, as is shown by FIG. 16.

If the tunneling effort is properly done, the front end of the tunneling device should emerge from the underlying soft tissues through the first incision site 300 then lying directly over the jugular vein. However, depending upon how far down the arm of the patient the secondary incision (blood inflow) site 310 is selected, as well as upon how direct a tunneling pathway 330 is made subcutaneously within the patient's arm, the use of a second tunneling device and additional small connecting incision may be necessary in order to connect the brachial and jugular incision sites 300, 310.

11. After the ascending tunnel and passageway 330 is made, remove the tunneling device via the venous incision site 300, leaving the tunneling sheath 100 in place within the formed subcutaneous tunnel passageway 330.

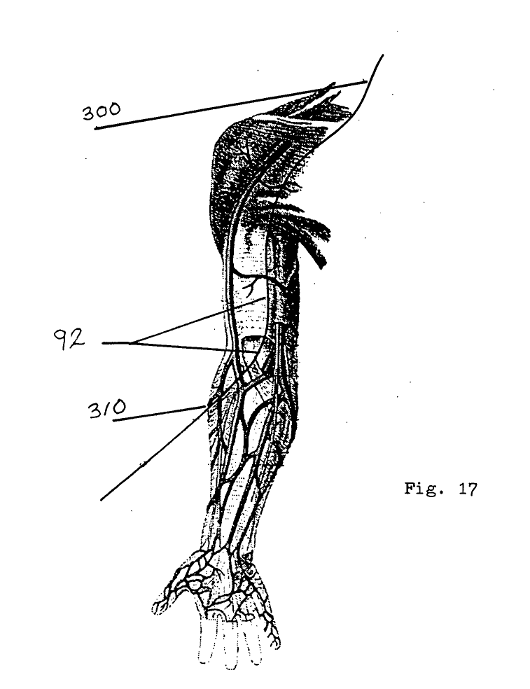

12. Pass the withdrawal strand 92 of the obturator 60 (then lying within the internal lumen of the endograft) through the tunneling sheath 100 and the subcutaneous passageway 330 from the first (venous) incision site 300 in the neck of the patient, until the withdrawl strand 92 of the obturator 60 emerges through the secondary incision site 310 lying over the brachial artery. This is illustrated by FIG. 17 (in which the subcutaneous passage is not shown).

13. Once the withdrawl strand 92 of the obturator 60 is secured, remove the tunneling sheath 100 by peeling back its plastic sides, thereby leaving only the obturator withdrawal strand 92 (still located in the distal conduit end 32 of the endograft 10) in the spatial volume of the subcutaneous tunnel 330.

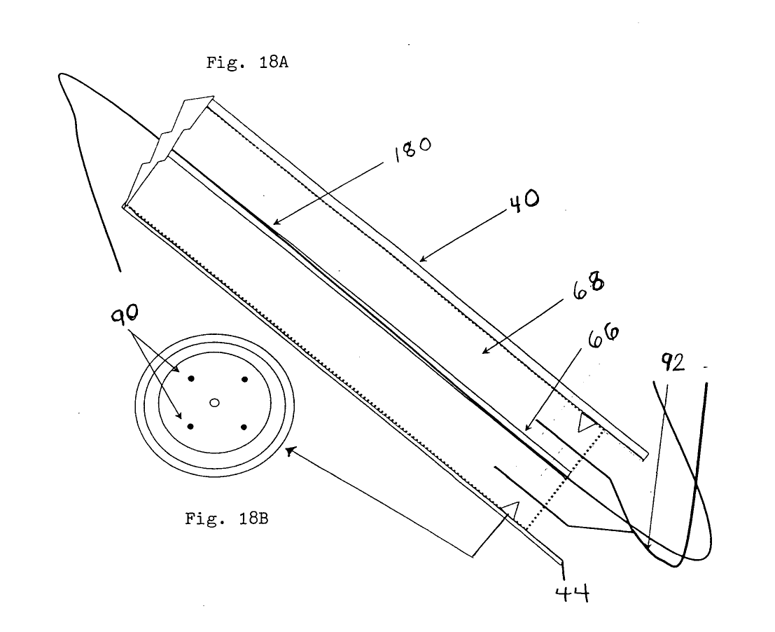

14. While securing the endograft 10 in place at the jugular vein site 300, withdraw the obturator 60 until it locks into the proximal conduit arm 40 and proximal conduit (blood inflow) end 44 of the endograft 10. Then, after removing any air, flush the interior and the exterior surfaces of the endograft with heparin/saline solution. This effort and result is shown by FIGS. 18A and 18B respectively.

15. Apply gentle traction to the obturator withdrawal strand 92 below the secondary incision site 310 above the brachial artery. Then, advance the obturator 60 and the entire proximate conduit arm 40 of the endograft 10 through the tunnel passageway 330 until the proximal conduit (blood inflow) end 44 visibly emerges through the secondary incision site 310 over the brachial artery. This action, in turn, will also cause the ribbed medial section 20 of the endograft 10 to be pulled into and through the first (venous) incision site 300.

16. Carefully manipulate the central ribbed portion 20 of the endograft 10 to insure that only a gentle, non-kinked and non-twisted curve is allowed to form as the article physically enters the first (venous) incision site 300 and traverses the skin at the base of the neck and upper shoulder area. Also, be sure to allow enough length and linear distance for the proximal conduit arm 40 such that it will lie in an non-stretched manner within the tunnel passageway 330. This is done by moving the proximal conduit arm 40 in abduction and/or adduction so that it does not foreshorten or create any tension within the spatial volume of the tunnel passageway 330.

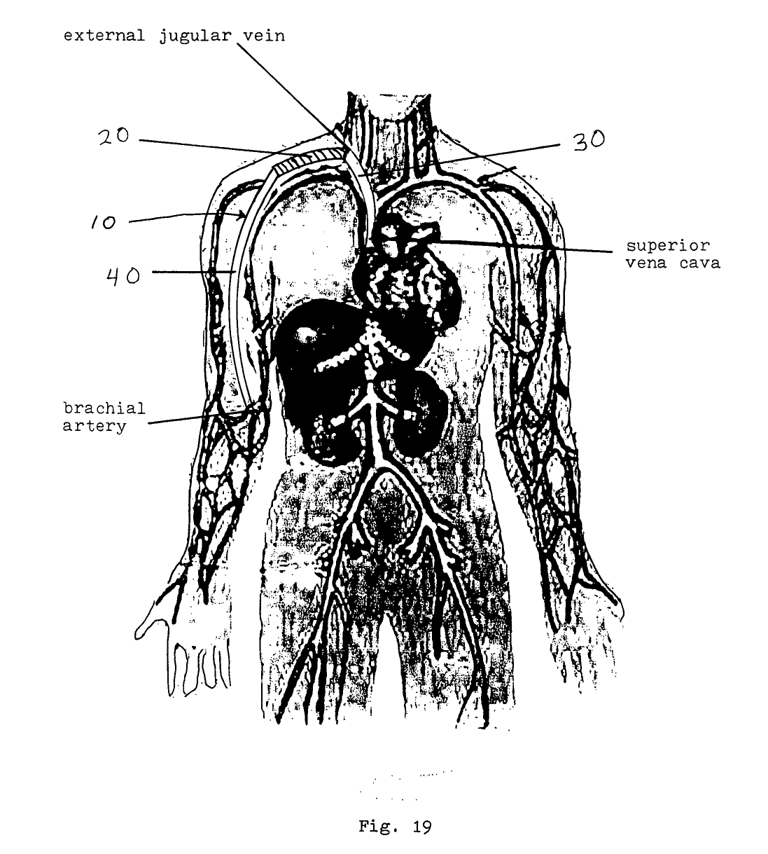

17. Remove the obturator 60 via the secondary incision site 310. Then, carefully measure and custom-cut the proximal conduit arm 40 at the proximal conduit end 44 to the appropriate length such that the sized arm 40 will rest directly over the brachial artery. The proximal conduit arm 40 thus is custom-sized in length and is now ready for direct surgical attachment (anastomosis) and fluid flow juncture to the brachial artery. This is shown by FIG. 19.

18. Complete the proximal (inflow) vascular anastamosis to the brachial artery in accordance with conventional surgical technique and medical fashion. Then, de-air the anastamosis; remove the atraumatic graft clamp; and allow blood from the brachial artery to flow through the attached proximal conduit end 44 and proximal conduit arm 40 into the ribbed medial section 20, and then into the distal conduit arm 30 previously positioned at the cavo-atrial junction in the patient's heart.

(iv) Completion of the Surgical Procedure:

15. The two small skin incisions 300, 310 [the venous site incision and the arterial site incision] each are irrigated with a prepared antimicrobial solution; and then are surgically closed in the conventional known and medically appropriate fashion. Standard post-operative follow˜up and care is then provided to the patient.

16. The subcutaneously inserted endograft can be used for dialysis access in approximately four weeks time after implantation. The repeated puncture of the ribbed medial section by dialysis needles (for hemodialysis purposes) is self-sealing and markedly limits the risk of hemorrhage.

### V. Critical Requirements of the Surgical Method

1. Precise Subject-Customized Sizing of the Distal and Proximal Conduit Arm Linear Lengths in Advance by the Surgeon:

The surgeon will size-customize each endograft according to the patient's anatomy and body habitus. The distal end of the prosthetic endograft will be positioned at the atrio-caval junction using the angiographic markers and fluoroscopy. Once that location is determined, the distance from that point to the percutaneous puncture in the neck will be measured. The surgeon will then cut the distal conduit arm of the endograft such that the distance from the beginning of the ribbed portion to the distal conduit end is exactly that measured length. The distal conduit arm of the endograft will then be inserted and positioned into the vein.

The ribbed portion will now begin as the endograft exits the neck incision. The subcutaneous tunnel passageway will exist down the arm and the endograft will be pulled through the tunnel sheath such that it will lie subcutaneously until it exits at the brachial artery incision site. The ribbed portion will be positioned in the area of the neck and shoulder subcutaneously and flexibly so that the endograft does not kink or bend in its course down to the arm. Just above the elbow level where the brachial artery has been identified and dissected free, the endograft will exit the subcutaneous tunnel passageway and be externally visble. Now the surgeon will position, measure and cut the proximal conduit arm such that a properly placed graft-to-artery anastamosis can be performed without kinking or bending and provide an unobstructed blood flow through the endograft.

2. Accurate Anatomic Placement of the Distal Conduit End at the Cavo-Atrial Junction:

Once the jugular vein has been percutaneously punctured, a guide wire will be inserted through the needle and into the right atrium. A 5Fr angiographic catheter with 1 cm radio-opaque markers will be threaded over the wire and positioned down the jugular vein and superior vena cava to the level of the atrio-caval junction. Using fluoroscopic guidance and intravascular contrast injections, that site will be accurately identified. Once the tip of the angiographic catheter is positioned at that junction, the distance from the atrio-caval junction to the jugular vein puncture site in the neck will be measured and that distance will now be used to cut the distal endograft to its proper length. The 5Fr catheter will be removed leaving the guide wire in place and the endograft and obturator assembly will now be threaded over the wire and positioned at the previously identified and measured atrio-caval junction. This anatomic position will again be confirmed with a fluoroscopic contrast injection.

3. The Absence of an Anastomosis at the Distal (Outflow) Conduit End:

After its proper positioning at the atrio-caval junction, the distal end of the endograft will be free floating within the lumen of the superior vena cava. There will be no need of any anastamosis; and normal venous return from the arm, neck and head will occur around the endograft. At the level of the jugular vein where the endograft enters, there will also be no anastamosis. Because of the low pressure venous system, the distensibility of the jugular vein and the fact that the graft entrance site into the jugular vein will be a tight fit because no surgical incision was made, there will be no need for any sutured anastamosis. The venous entry site will seal naturally around the endograft.

4. The Need for an Anastomosis at the Proximal (Inflow) Conduit End:

Once the endograft has been passed through the tunneled passageway subcutaneously through the neck and down the arm, it will exit the tunnel passageway through the surgically made skin incision at the brachial artery site. A point of intended attachment will be chosen on the brachial artery; and the endograft will then be measured and custom-cut so that a standard sutured vascular anastamosis can be performed. This will be an end-to-side vascular anastamosis (end of the endograft sutured to the side of the brachial artery). Once completed, arteriovenous flow will be established from the brachial artery through the endograft interior and into the right atrium.

### VI. Medical Precautions and Potential Complications of the Surgical Method

1. The medical precautions and potential complications will be those of any surgically created A-V vascular access. In general those complications include bleeding at the percutaneous entrance site in the neck, at the surgical incision site, and at the vascular anastamosis in the arm. Additionally, bleeding can occur along and through the subcutaneous tunnel passageway because of potentially disrupted small vessels while creating the tunnel in a blunt manner. Also, thrombosis of the endograft can occur such that flow through the A-V endograft will cease. Furthermore, thrombosis or injury can occur to the native vessels involved, specifically the brachial artery and the jugular vein and/or the superior vena cava.

Infection can occur at any of these sites, the bacteria being introduced at the time of the surgery or at a later date while using the A-V endograft for dialysis. Re-operation may be necessary at various times because of bleeding, thrombosis, anatomic malposition, or kinking of the endograft; and removal may become necessary because of infection or a revision of the graft owing to any or all of the above-mentioned problems.

A “steal” syndrome may also occur in the arm. This is a phenomenon whereby after the A-V endograft has been created and blood flow established, the endograft itself may “steal” blood flow from the distal extremity such that arterial insufficiency is experienced and complications thereof. While uncommon, it can occur and be seen with any surgically created A-V connection.

2. Precautions which can be taken to avoid such complications are also standard; and the same set of precautions that would be performed in any conventionally known surgically created A-V graft for vascular access. These precautions include making sure of the distal endograft placement at the atrio-caval junction using the steps and methods outlined. Additionally, one must make sure that any bleeding or bleeding problems are addressed at the time of operation and properly corrected.

Thrombosis may be avoided by strict attention to prevention of kinking of the endograft in its course from the atrio-caval junction all the way to the brachial artery anastamosis. Identifying and documenting free flow at the end of the procedure using fluoroscopic contrast imaging will also be a preventative step; as well as liberal use of these same methods throughout the procedure to identify proper vessels, locations and configurations.

Infection can be prevented by standard sterile surgical technique as well as the use of pre-operative and post-operative antibiotics in a prophylactic manner. While the “steal” syndrome may not be able to be predicted or prevented, identifying those individuals who may be at greater risk for such a complication is useful, so that an awareness of said syndrome is present. Finally, precise and accurate identification, placement, creation and performance of aforementioned steps will be the best preventative measures to avoid complications and problems with this method. As stated previously herein, such potential complications and problems are no different or greater in number than the standard surgical vascular access creation that is performed at present.

### VII. Other Potential Therapeutic Uses and Future Clinical Applications in Addition to Hemodialysis

Clearly hemodialysis is the present and primary focus of the present invention. Nevertheless, there are other clinical applications and therapeutic uses which are envisioned and are deemed to be available at the present time. Additionally, it is expected that there are also a number of future conditions and endeavors which will use this apparatus and methodology to marked advantage.

For these reasons, a listing of present and immediate possible uses for the vascular access provided by the present invention is given by Table 2; and a listing of envisioned clinical applications in the foreseeable future is given by Table 3 below.

TABLE 2
Present and immediate possible uses

Plasmapheresis;
Erythropheresis;
Leucopheresis;
Plateletpheresis;
Long-term instillation of antibiotics;
Chemotherapy treatment; and
Long-term or permanent parenteral hyperalimentation (nutritional support)

TABLE 3
Envisioned clinical applications in the foreseeable future

Hyperthermic regional chemotherapy;
Monoclonal antibody therapy;
Hepatic hemo-detoxification;
Microsphere-directed radio-tagged, or chemo-tagged, antibody therapy;
Bone marrow transplantation; and
Hypothermic circulatory arrest and/or suspended animation

The present invention is not to be restricted in form nor limited in scope except by the claims appended hereto:

1. A subject-customized prosthetic endograft suitable as a durable vascular access for the carrying of flowing blood and serviceable after surgical insertion into a particular subject suffering from a clinically recognized condition, said subject-customized prosthetic endograft comprising:
a flexible, elongated hollow tube construct formed of at least one durable and biocompatible material and comprised of
(i) a hollow ribbed medial section having a predetermined length, external diameter size, tubular wall thickness, and internal lumen diameter, and whose tubular wall can be repeatedly penetrated on-demand by syringe needles;
(ii) a hollow distal conduit arm having two open ends, one open end terminating as a discrete distal conduit end and the other open end being integrally joined to and in fluid flow communication with said ribbed medial section, said distal conduit arm being of predetermined external diameter size, tubular wall thickness, and internal lumen diameter, and having a subject-customized linear length which is to be custom-sized by a surgeon such that after in-vivo insertion of said sized distal conduit arm into a pre-chosen vein, said distal conduit end will float freely within the vein and anatomically lie adjacent to the cavo-atrial junction of the heart in the particular subject; and
(iii) a hollow proximal conduit arm having two open ends, one end terminating as a discrete proximal conduit end and the other end being integrally joined to and in fluid flow communication with said ribbed medial section, said proximal conduit arm being of predetermined external diameter size, tubular wall thickness, and internal lumen diameter, and having a subject-customized linear length which is to be custom-sized by the surgeon such that said sized proximal conduit arm can be subcutaneously positioned over its entire sized length within the upper limb of the particular subject in-vivo, and said proximal conduit end can be surgically joined to and anastomosed at a pre-selected anatomic site with a pre-chosen artery in the upper limb of the particular subject.

2. A subject-customized prosthetic endograft suitable for the carrying of flowing blood and serviceable after surgical insertion as a durable vascular access for long-term hemodialysis in a particular subject afflicted with end stage renal disease, said subject-customized prosthetic endograft comprising:
a flexible, elongated hollow tube construct formed of at least one durable and biocompatible material and comprised of
(i) a hollow ribbed medial section having a predetermined length, external diameter size, tubular wall thickness, and internal lumen diameter, and whose tubular wall can be repeatedly penetrated on-demand by hemodialysis needles;
(ii) a hollow distal conduit arm having two open ends, one open end terminating as a discrete distal conduit end and the other open end being integrally joined to and in fluid flow communication with said ribbed medial section, said distal conduit arm being of predetermined external diameter size, tubular wall thickness, and internal lumen diameter, and having a subject-customized linear length which is to be custom-sized by a surgeon such that after in-vivo insertion of said sized distal conduit arm into a pre-chosen vein, said distal conduit end will float freely within the vein and anatomically lie adjacent to the cavo-atrial junction of the heart in the particular subject; and
(iii) a hollow proximal conduit arm having two open ends, one end terminating as a discrete proximal conduit end and the other end being integrally joined to and in fluid flow communication with said ribbed medial section, said proximal conduit arm being of predetermined external diameter size, tubular wall thickness, and internal lumen diameter, and having a subject-customized linear length which is to be custom-sized by the surgeon such that said sized proximal conduit arm can be subcutaneously positioned over its entire sized length within the upper limb of the particular subject in-vivo, and said proximal conduit end can be surgically joined to and anastomosed at a pre-selected anatomic site with a pre-chosen artery in the upper limb of the particular subject.

3. The subject-customized prosthetic endograft as recited in claim 1 or 2 further comprising a plurality of radiographic markers disposed at pre-measured intervals on the exterior of said distal conduit arm.

4. The subject-customized prosthetic endograft as recited in claim 1 or 2 wherein said durable and biocompatible material is chemically formulated as a type of polytetrafluoroethylene (PTFE).

5. The subject-customized prosthetic endograft as recited in claim 1 or 2 wherein said durable and biocompatible material is a substance selected from the group consisting of polyethylene terephthalate fibers and fabrics, a multi-layered and self-sealing polyurethane, a bioartificial matter derived from mesenteric vein, and a cryopreserved allograft material from which cellular elements have been removed using antigen reduction technology.

6. A surgical prosthetic endograft insertion kit whose components are to be used to create a durable vascular access suitable for long-term hemodialysis in a particular subject afflicted with end stage renal disease, said surgical prosthetic endograft insertion kit comprising:
(a) a subject-customized prosthetic endograft suitable for the carrying of flowing blood, which is configured as a flexible, elongated hollow tube and is constructed of at least one durable and biocompatible material, said prosthetic endograft comprising
(i) a hollow ribbed medial section having a predetermined length, external diameter size, tubular wall thickness, and internal lumen diameter, and whose tubular wall can be repeatedly penetrated on-demand by dialysis needles,
(ii) a hollow distal conduit arm having two open ends, one end terminating as a discrete distal conduit end and the other end being integrally joined to and in fluid flow communication with said ribbed medial section, said distal conduit arm being of predetermined external diameter size, tubular wall thickness, and internal lumen diameter, and having a subject-customized linear length which is to be custom-sized by a surgeon such that after in-vivo insertion of said sized distal conduit arm into a pre-chosen vein in the particular subject, said distal conduit end will float freely within the vein and anatomically lie adjacent to the cavo-atrial junction of the heart in the particular subject,
(iii) a hollow proximal conduit arm having two open ends, one end terminating as a discrete proximal conduit end and the other end being integrally joined to and in fluid flow communication with said ribbed medial section, said proximal conduit arm being of predetermined external diameter size, tubular wall thickness, and internal lumen diameter, and having a subject-customized linear length which is to be custom-sized by a surgeon such that said sized proximal conduit arm can be subcutaneously positioned over its entire sized length within the upper limb in a particular subject, and said proximal conduit end can be surgically joined to and anastomosed at a pre-selected anatomic site with a pre-chosen artery in the upper limb of the particular subject;
(b) a flexible vascular graft obturator formed of durable material and having pre-determined dimensions and configuration, said vascular graft obturator having a tapered conical distal end, a rounded proximal end, a central lumen able to accommodate the passage of a cable therethrough, and a withdrawl cable whose overall length passes through said central lumen;
(c) a tunneling apparatus comprising
a peel-away tunneling sheath of determinable length and volume, and
a central, conical-ended tunneling tool which can be locked into said tunneling sheath on-demand; and
(d) Seldinger technique workpieces comprising
a Seldinger needle of specific gauge,
a vein dilator of known linear length and diameter which has a plurality of measurement markers over its length, and
a guide wire of specified girth and length.

7. A surgical prosthetic endograft insertion kit whose components are to be used to create a durable vascular access, said surgical prosthetic endograft insertion kit comprising:
(a) a subject-customized prosthetic endograft suitable for the carrying of flowing blood, which is configured as a flexible, elongated hollow tube and is constructed of at least one durable and biocompatible material, said prosthetic endograft comprising
(i) a hollow ribbed medial section having a predetermined length, external diameter size, tubular wall thickness, and internal lumen diameter, and whose tubular wall can be repeatedly penetrated on-demand by syringe needles,
(ii) a hollow distal conduit arm having two open ends, one end terminating as a discrete distal conduit end and the other end being integrally joined to and in fluid flow communication with said ribbed medial section, said distal conduit arm being of predetermined external diameter size, tubular wall thickness, and internal lumen diameter, and having a subject-customized linear length which is to be custom-sized by a surgeon such that after in-vivo insertion of said sized distal conduit arm into a pre-chosen vein in the particular subject, said distal conduit end will float freely within the vein and anatomically lie adjacent to the cavo-atrial junction of the heart in the particular subject,
(iii) a hollow proximal conduit arm having two open ends, one end terminating as a discrete proximal conduit end and the other end being integrally joined to and in fluid flow communication with said ribbed medial section, said proximal conduit arm being of predetermined external diameter size, tubular wall thickness, and internal lumen diameter, and having a subject-customized linear length which is to be custom-sized by a surgeon such that said sized proximal conduit arm can be subcutaneously positioned over its entire sized length within the upper limb in a particular subject, and said proximal conduit end can be surgically joined to and anastomosed at a pre-selected anatomic site with a pre-chosen artery in the upper limb of the particular subject;
(b) a flexible vascular graft obturator formed of durable material and having pre-determined dimensions and configuration, said vascular graft obturator having configured distal and proximal ends, a central lumen able to accommodate the passage of a cable therethrough, and a withdrawl cable whose linear length passes through said central lumen;
(c) a tunneling apparatus comprising
a peel-away tunneling sheath of determinable linear length and spatial volume, and
a central, conical-ended tunneling tool which can be locked into said tunneling sheath on-demand; and
(d) Seldinger technique workpieces comprising
a Seldinger needle of specific gauge,
a vein dilator of known linear length and diameter which has a plurality of measurement markers over its length, and
a guide wire of specified girth and length.

8. A surgical method for creating a durable vascular access in a particular subject suffering from a clinically recognized condition, said surgical method comprising the steps of:
(a) obtaining a subject-customized prosthetic endograft configured as a flexible, elongated hollow tube and constructed of at least one durable and biocompatible material, said prosthetic graft article comprising
(i) a hollow ribbed medial section having a predetermined length, external diameter size, tubular wall thickness, and internal lumen diameter, and whose tubular wall can be repeatedly penetrated on-demand by syringe needles,
(ii) a hollow distal conduit arm having two open ends, one end terminating as a discrete distal conduit end and the other end being integrally joined to and in fluid flow communication with said ribbed medial section, said distal conduit arm being of predetermined external diameter size, tubular wall thickness, and internal lumen diameter, and having a subject-customized linear length which is custom-sized by the surgeon such that after in-vivo insertion of said sized distal conduit arm into a pre-chosen vein in the particular subject, said distal conduit end will float freely within the vein and anatomically lie adjacent to the cavo-atrial junction of the heart in the particular subject,
(iii) a hollow proximal conduit arm having two open ends, one end terminating as a discrete proximal conduit end and the other end being integrally joined to and in fluid flow communication with said ribbed medial section, said proximal conduit arm being of predetermined external diameter size, tubular wall thickness, and internal lumen diameter, and having a subject-customized linear length which is custom-sized by the surgeon such that said sized proximal conduit arm can be subcutaneously positioned over its entire sized length within the upper limb in a particular subject, and said proximal conduit end can be surgically joined to and anastomosed at a pre-selected anatomic site with a pre-chosen artery in the upper limb of the particular subject;
(b) percutaneously passing said custom-sized distal conduit arm of said prosthetic graft article through a first insertion site at a pre-selected anatomic position into the internal lumen of the pre-chosen vein in the particular subject, whereby said custom-sized distal conduit arm comes to rest entirely within the lumen of the pre-chosen vein, and whereby said distal conduit end floats freely and anatomically lies within the pre-chosen vein adjacent to the cavo-atrial junction of the heart in the particular subject;
(c) creating a second insertion site at a second pre-selected anatomic position in the upper limb of the particular subject to gain access to a pre-chosen artery in the upper limb of the particular subject;
(d) surgically forming a subcutaneous tunnel and open passageway within the upper limb which extends upwardly from said second insertion site and terminates adjacent to the first insertion site in the neck/shoulder of the particular patient, said formed subcutaneous tunnel and open passageway being substantially parallel to the anatomic location of the pre-chosen artery within the upper limb;
(e) passing said proximal conduit arm of said prosthetic endograft into and through the length of said subcutaneous tunnel and open passageway such that said custom-sized proximal conduit end lies adjacent to said second insertion site on the upper limb of the particular patient;
(f) introducing said ribbed medial section of said prosthetic endograft through said first insertion site such said ribbed medial section lies subcutaneously adjacent to said open passageway and subcutaneous tunnel; and
(g) joining said custom-sized proximal conduit end to said pre-chosen artery in the upper limb of the particular subject.

9. The method as recited in claim 8 wherein the clinically recognized condition is one selected from the group consisting of plasmapheresis, erythropheresis, leucopheresis, platletpheresis, long-term instillation of antibiotics, chemotherapy treatment, and parenteral hyperalimentation.

10. The method as recited in claim 8 wherein the clinically recognized condition is one selected from the group consisting of hyperthermic region chemotherapy, monoclonal antibody therapy, hepatic hemo-detoxification, micro-sphere-directed antibody therapy, bone marrow transplantation, hypothermic circulatory arrest, and suspended animation.

11. A surgical method for creating a durable vascular access suitable for long-term hemodialysis in a particular subject afflicted with end stage renal disease, said surgical method comprising the steps of:
(α) creating a first insertion site at a pre-selected anatomic position in the neck/shoulder of the particular subject to percutaneously puncture a pre-chosen vein;
(β) preparing a subject-customized prosthetic endograft configured as a flexible, elongated hollow tube and constructed of at least one durable and biocompatible material, said prosthetic graft article comprising
(i) a hollow ribbed medial section having a predetermined length, external diameter size, tubular wall thickness, and internal lumen diameter, and whose tubular wall can be repeatedly penetrated on-demand by dialysis needles,
(ii) a hollow distal conduit arm having two open ends, one end terminating as a discrete distal conduit end and the other end being integrally joined to and in fluid flow communication with said ribbed medial section, said distal conduit arm being of predetermined external diameter size, tubular wall thickness, and internal lumen diameter, and having a subject-customized linear length which is custom-sized by the surgeon such that after in-vivo insertion of said sized distal conduit arm into a pre-chosen vein in the particular subject, said distal conduit end will float freely within the vein and anatomically lie adjacent to the cavo-atrial junction of the heart in the particular subject,
(iii) a hollow proximal conduit arm having two open ends, one end terminating as a discrete proximal conduit end and the other end being integrally joined to and in fluid flow communication with said ribbed medial section, said proximal conduit arm being of predetermined external diameter size, tubular wall thickness, and internal lumen diameter, and having a subject-customized linear length which is custom-sized by the surgeon such that said sized proximal conduit arm can be subcutaneously positioned over its entire sized length within the upper limb in a particular subject, and said proximal conduit end can be surgically joined to and anastomosed at a pre-selected anatomic site with a pre-chosen artery in the upper limb of the particular subject;
(γ) percutaneously passing said custom-sized distal conduit arm of said prosthetic endograft through said insertion site into the internal lumen of the pre-chosen vein in the particular subject, whereby said custom-sized distal conduit arm comes to rest entirely within the lumen of the pre-chosen vein, and whereby said distal conduit end floats freely and anatomically lies within the pre-chosen vein adjacent to the cavo-atrial junction of the heart in the particular subject;
(δ) creating a second insertion site at a second pre-selected anatomic position in the upper limb of the particular subject to gain access to a pre-chosen artery in the upper limb of the particular subject;
(ε) mobilizing a segment of the accessed pre-chosen artery in the upper limb of the particular subject;
(ζ) surgically forming a subcutaneous tunnel and open passageway within the upper limb which extends upwardly from said second insertion site and terminates adjacent to the first insertion site in the neck/shoulder of the particular patient, said formed subcutaneous tunnel and open passageway being substantially parallel to the anatomic location of the pre-chosen artery within the upper limb;
(η) passing said proximal conduit arm of said prosthetic endograft into and through the length of said subcutaneous tunnel and open passageway such that said custom-sized proximal conduit end lies adjacent to said second insertion site on the upper limb of the particular patient;
(θ) introducing said ribbed medial section of said prosthetic endograft through said first insertion site such said ribbed medial section lies subcutaneously adjacent to said open passageway and subcutaneous tunnel; and
(ι) joining and anastomosing said custom-sized proximal conduit end to said mobilized segment of the pre-chosen artery in the upper limb of the particular subject; and
(κ) surgically closing said first and second insertion sites.

12. An in-vivo durable vascular access for the carrying of flowing blood and serviceable for a particular subject suffering from a clinically recognized condition, said durable vascular access comprising:
a custom-sized and subcutaneously positioned endograft comprised of
a flexible, elongated hollow tube construct formed of at least one durable and biocompatible material and comprised of
(i) a hollow ribbed medial section having a predetermined length, external diameter size, tubular wall thickness, and internal lumen diameter, and whose tubular wall can be repeatedly penetrated on-demand by syringe needles;
(ii) a hollow distal conduit arm having two open ends, one open end terminating as a discrete distal conduit end and the other open end being integrally joined to and in fluid flow communication with said ribbed medial section, said distal conduit arm being of predetermined external diameter size, tubular wall thickness, and internal lumen diameter, and having a subject-customized linear length whereby said sized distal conduit arm lies in a pre-chosen vein, and wherein said distal conduit end will floats freely within the vein and anatomically lies adjacent to the cavo-atrial junction of the heart in the particular subject; and
(iii) a hollow proximal conduit arm having two open ends, one end terminating as a discrete proximal conduit end and the other end being integrally joined to and in fluid flow communication with said ribbed medial section, said proximal conduit arm being of predetermined external diameter size, tubular wall thickness, and internal lumen diameter, and having a subject-customized linear length whereby said sized proximal conduit arm is subcutaneously positioned over its entire sized length within the upper limb of the particular subject in-vivo, and wherein said proximal conduit end is surgically joined to a pre-chosen artery in the upper limb of the particular subject.

13. The in-vivo durable vascular access as recited in claim 12 where the clinical condition is hemodialysis.

14. The in-vivo durable vascular access as recited in claim 12 where the clinical condition is one selected from the group consisting of plasmapheresis, erythropheresis, leucopheresis, platletpheresis, long-term instillation of antibiotics, chemotherapy treatment, and parenteral hyperalimentation.

15. The in-vivo durable vascular access as recited in claim 12 where the clinical condition is one selected from the group consisting of hyperthermic region chemotherapy, monoclonal antibody therapy, hepatic hemo-detoxification, micro-sphere-directed antibody therapy, bone marrow transplantation, hypothermic circulatory arrest, and suspended animation.
